SEQUENCE LISTING (63 sequences)

SEQ ID NO: 1 - DNA, 23 bp, synthetic construct
  source 1..23: mol_type = other DNA
ccttggcacacccgagaattcca

SEQ ID NO: 2 - DNA, 25 bp, synthetic construct
  source 1..25: mol_type = other DNA
actgacactggaataggaatgtgat

SEQ ID NO: 3 - DNA, 23 bp, synthetic construct
  source 1..23: mol_type = other DNA
ctccaaggagtaagaaaccctgg

SEQ ID NO: 4 - DNA, 24 bp, synthetic construct
  source 1..24: mol_type = other DNA
ctgccctctgggatggatctatag

SEQ ID NO: 5 - DNA, 25 bp, synthetic construct
  source 1..25: mol_type = other DNA
ttttaatttctgaatggcccaggtc

SEQ ID NO: 6 - DNA, 25 bp, synthetic construct
  source 1..25: mol_type = other DNA
tcctgcatgtttttactgatgttcg

SEQ ID NO: 7 - DNA, 25 bp, synthetic construct
  source 1..25: mol_type = other DNA
ctggaatttgcagaatgactggaag

SEQ ID NO: 8 - DNA, 25 bp, synthetic construct
  source 1..25: mol_type = other DNA
atcatatgccaaaccctctgtactt

SEQ ID NO: 9 - DNA, 25 bp, synthetic construct
  source 1..25: mol_type = other DNA
actgacaaagctgtactgagtatga

SEQ ID NO: 10 - DNA, 25 bp, synthetic construct
  source 1..25: mol_type = other DNA
gtctccattctgttagagaccatga

SEQ ID NO: 11 - DNA, 23 bp, synthetic construct
  source 1..23: mol_type = other DNA
atacagggtggcactgaaaactg

SEQ ID NO: 12 - DNA, 25 bp, synthetic construct
  source 1..25: mol_type = other DNA
actgcctcctcctctcttgtaaata

SEQ ID NO: 13 - DNA, 25 bp, synthetic construct
  source 1..25: mol_type = other DNA
atgaatgtaactcagccaagaaacg

SEQ ID NO: 14 - DNA, 25 bp, synthetic construct
  source 1..25: mol_type = other DNA
tctaccttgtgaaacaattgtcagc

SEQ ID NO: 15 - DNA, 25 bp, synthetic construct
  source 1..25: mol_type = other DNA
gtggtctagataactacactgcctg

SEQ ID NO: 16 - DNA, 25 bp, synthetic construct
  source 1..25: mol_type = other DNA
atttctagtacggcacatgagatga

SEQ ID NO: 17 - DNA, 26 bp, synthetic construct
  source 1..26: mol_type = other DNA
tggaccaagaatggtaagaacatttg

SEQ ID NO: 18 - DNA, 25 bp, synthetic construct
  source 1..25: mol_type = other DNA
atagaaagcatgtaggagactggtg

SEQ ID NO: 19 - DNA, 25 bp, synthetic construct
  source 1..25: mol_type = other DNA
gacgacgatatctttgaaaagcact

SEQ ID NO: 20 - DNA, 23 bp, synthetic construct
  source 1..23: mol_type = other DNA
gaagtgagactgccaggacatga

SEQ ID NO: 21 - DNA, 25 bp, synthetic construct
  source 1..25: mol_type = other DNA
ccctcaactgttcactcttagaact

SEQ ID NO: 22 - DNA, 46 bp, synthetic construct
  source 1..46: mol_type = other DNA
ccttggcacccgagaattccaactgacactggaataggaatgtgat

SEQ ID NO: 23 - DNA, 44 bp, synthetic construct
  source 1..44: mol_type = other DNA
ccttggcacccgagaattccactccaaggagtaagaaaccctgg

SEQ ID NO: 24 - DNA, 45 bp, synthetic construct
  source 1..45: mol_type = other DNA
ccttggcacccgagaattccactgccctctgggatggatctatag

SEQ ID NO: 25 - DNA, 46 bp, synthetic construct
  source 1..46: mol_type = other DNA
ccttggcacccgagaattccattttaatttctgaatggcccaggtc

SEQ ID NO: 26 - DNA, 46 bp, synthetic construct
  source 1..46: mol_type = other DNA
ccttggcacccgagaattccatcctgcatgtttttactgatgttcg

SEQ ID NO: 27 - DNA, 46 bp, synthetic construct
  source 1..46: mol_type = other DNA
ccttggcacccgagaattccactggaatttgcagaatgactggaag

SEQ ID NO: 28 - DNA, 46 bp, synthetic construct
  source 1..46: mol_type = other DNA
ccttggcacccgagaattccaatcatatgccaaaccctctgtactt

SEQ ID NO: 29 - DNA, 46 bp, synthetic construct
  source 1..46: mol_type = other DNA
ccttggcacccgagaattccaactgacaaagctgtactgagtatga

SEQ ID NO: 30 - DNA, 46 bp, synthetic construct
  source 1..46: mol_type = other DNA
ccttggcacccgagaattccagtctccattctgttagagaccatga

SEQ ID NO: 31 - DNA, 44 bp, synthetic construct
  source 1..44: mol_type = other DNA
ccttggcacccgagaattccaatacagggtggcactgaaaactg

SEQ ID NO: 32 - DNA, 46 bp, synthetic construct
  source 1..46: mol_type = other DNA
ccttggcacccgagaattccaactgcctcctcctctcttgtaaata

SEQ ID NO: 33 - DNA, 46 bp, synthetic construct
  source 1..46: mol_type = other DNA
ccttggcacccgagaattccaatgaatgtaactcagccaagaaacg

SEQ ID NO: 34 - DNA, 46 bp, synthetic construct
  source 1..46: mol_type = other DNA
ccttggcacccgagaattccatctaccttgtgaaacaattgtcagc

SEQ ID NO: 35 - DNA, 46 bp, synthetic construct
  source 1..46: mol_type = other DNA
ccttggcacccgagaattccagtggtctagataactacactgcctg

SEQ ID NO: 36 - DNA, 46 bp, synthetic construct
  source 1..46: mol_type = other DNA
ccttggcacccgagaattccaatttctagtacggcacatgagatga

SEQ ID NO: 37 - DNA, 47 bp, synthetic construct
  source 1..47: mol_type = other DNA
ccttggcacccgagaattccatggaccaagaatggtaagaacatttg

SEQ ID NO: 38 - DNA, 46 bp, synthetic construct
  source 1..46: mol_type = other DNA
ccttggcacccgagaattccaatagaaagcatgtaggagactggtg

SEQ ID NO: 39 - DNA, 46 bp, synthetic construct
  source 1..46: mol_type = other DNA
ccttggcacccgagaattccagacgacgatatctttgaaaagcact

SEQ ID NO: 40 - DNA, 44 bp, synthetic construct
  source 1..44: mol_type = other DNA
ccttggcacccgagaattccagaagtgagactgccaggacatga

SEQ ID NO: 41 - DNA, 46 bp, synthetic construct
  source 1..46: mol_type = other DNA
ccttggcacccgagaattccaccctcaactgttcactcttagaact

SEQ ID NO: 42 - DNA, 209 bp, synthetic construct
  source 1..209: mol_type = other DNA
actgacactggaataggaatgtgatgggcgtcgcaccctctgtaaatgtgggaatgtttgtaacttgtgtttgtatctaatgtcgatccccttaggtggttgtactataattcatttttgttgtaaacttcagcctaagataaatgtacatctgcttttgtatgcactcattaaacattgtaacagaccaaagatggtgtactaattgc

SEQ ID NO: 43 - DNA, 191 bp, synthetic construct
  source 1..191: mol_type = other DNA
ctccaaggagtaagaaaccctggaccacccaccccagcaaggacactgagcaagagaggccctatcccaactcggcccccaacactgagcatctccctcacaatttccatcccagacccccataataacaggaggggcctagggagccctccctactctcttgaataccatcaataaagttcgctgcaccc

SEQ ID NO: 44 - DNA, 201 bp, synthetic construct
  source 1..201: mol_type = other DNA
ctgccctctgggatggatctatagatggactttccaagctctcagaagctgagagcatctccagtttatcaattgagcccattgttcttagctctctcccacatcataaaccttctttctctgaacagaaaagagctctctttgtactcagatcagctaaaaattaaaatacagcagtgtaaacaagaaattttcttacaa

SEQ ID NO: 45 - DNA, 171 bp, synthetic construct
  source 1..171: mol_type = other DNA
ttttaatttctgaatggcccaggtctgaggcctcccttttttttgtccccccaacttgatgtatgaaggctttggtctccctgggagggggttgaggtgttgaggcagccagggctggcctgtacactgacttgagaccaataaaagtgcacaccttaccttacacaaaca

SEQ ID NO: 46 - DNA, 176 bp, synthetic construct
  source 1..176: mol_type = other DNA
tcctgcatgtttttactgatgttcgtgctggctgccctcagccctgagtctgggagaggctttggtgcctcgggtcagacttgggtgctccatggtagtggagcccttaaatgctttgtatattttctctattagatctcttttcagaagtgtctgtagaaaattaaaaaaaaaca

SEQ ID NO: 47 - DNA, 184 bp, synthetic construct
  source 1..184: mol_type = other DNA
ctggaatttgcagaatgactggaagtggggaaagtcactgtccaacttatcatccagcccctccttccccaaagatcaatatggcatgtaatatttaaagaaaacaggagatttgttcattctggaaaacaatgctcattatgtgacaataaactttatctcagtgtgactttggtgccaacaa

SEQ ID NO: 48 - DNA, 204 bp, synthetic construct
  source 1..204: mol_type = other DNA
atcatatgccaaaccctctgtacttctcattacttggatgcagttactcatctttggtctatcacaacataagtgacatactttccttttggtaaagcaaagaggcctaattgaagtctgtcactgtgcccaatgcttagcaattctcacccccaaccctgtggctacttctgcttttgttacttttactaaaaataaaaaact

SEQ ID NO: 49 - DNA, 187 bp, synthetic construct
  source 1..187: mol_type = other DNA
actgacaaagctgtactgagtatgaccatatgatattaagtcgagcagaggtcccaggaaccacaaggccaaccctccaagcatcttctcgcagtacctttgttttcaccaacctctcttgtcatttgttgtgtcctaatgctacttctgaagatagctgcaccaataaaatctatggcctgtggtt

SEQ ID NO: 50 - DNA, 182 bp, synthetic construct
  source 1..182: mol_type = other DNA
gtctccattctgttagagaccatgaagcagtattgtttaacataagttgtactgttaagtttggcttcatgggtgtaaacaccaatggtctgtcagtgtctaagactctggatactgcaagctccgtccggtgcatttgttcaggtaaaatctgtgcaataaaataacaaactgtctccaaa

SEQ ID NO: 51 - DNA, 208 bp, synthetic construct
  source 1..208: mol_type = other DNA
atacagggtggcactgaaaactgctgaagcccacagccctcagccccgaaggctcagcggccagtaccagcggccttgctgtggttgtgtgtctgcgtccctggcacacttgggcaggcagctcagtcactctgaacttgcttctcttctgtcttgtgagtgtgctgcttgagacaatgaaaaataaagctgtgttcctaccccttca

SEQ ID NO: 52 - DNA, 172 bp, synthetic construct
  source 1..172: mol_type = other DNA
actgcctcctcctctcttgtaaatacatgcatttgtacagtgggccctgttcttgtgaagtccatctccatggtcattagacctgccactctgaaccgcatgtgactcccccatgctcttggtctcccaggcccctgctatagccagagatcaataaagaagggagaccggc

SEQ ID NO: 53 - DNA, 200 bp, synthetic construct
  source 1..200: mol_type = other DNA
atgaatgtaactcagccaagaaacgtgttgctaagatacaatcctcagtgttctctgtatgtatatttctgtatataccacatgttacagcctgcatgagcttcctcacaccaagcccagccggcactgagcatgagatgctgttacatgtagacaaaggactgagatgttctcaataaagactaagacgtttcactatg

SEQ ID NO: 54 - DNA, 196 bp, synthetic construct
  source 1..196: mol_type = other DNA
tctaccttgtgaaacaattgtcagccctttggtgcctatccttctaaatatttctctatatctgtgttcctagattagaaatatgtatagacgaaagtgatcaaatagaagtgttgttctatatgctgtattttttcaccaaaacgtatgttgtggccttctttgtcaataaatatatacatatatgtcagcatct

SEQ ID NO: 55 - DNA, 193 bp, synthetic construct
  source 1..193: mol_type = other DNA
gtggtctagataactacactgcctgaaagctgtgattttagggtctgtgttcttttgagtcttgaagctcagtattggtctcttatggctatgttgttatcaatagtttgttacctcatctctcctgacgaaacatcaataaatgcttatttgtatataaatataataaacccgtgaccccaactgcacaatg

SEQ ID NO: 56 - DNA, 149 bp, synthetic construct
  source 1..149: mol_type = other DNA
atttctagtacggcacatgagatgagtcactgcctttttttctatggtgtacgacagttagagatgctgatttttttttcctgataaattctttctttaagaaagacaattttaatgtttacaacaataaaccacgtaaatgaacagaa

SEQ ID NO: 57 - DNA, 228 bp, synthetic construct
  source 1..228: mol_type = other DNA
tggaccaagaatggtaagaacatttgtatttttgaaaaaattgggagacacgggaaacagatattttatagcaaggcaaaataaaataaatatgtttgtcactaacaatacgttggcagtcatgtcattaaccaaactgtgtgcatgtgtcatttttctcttacgaagatttcttctgtttccagtttcctggattcagatatttaattaaagtttccataatgcttc

SEQ ID NO: 58 - DNA, 206 bp, synthetic construct
  source 1..206: mol_type = other DNA
atagaaagcatgtaggagactggtgatgtgttgacctttttaaaaaaaaacatatgtatatgtgtgtatatatatatacacacatatacatatatatgtgtatgtatgtacgtatgtatatatatatgaccaaaagaaaagaagagcacaagctgtctgaaccacaggttcttttatgtgtgtctaaataaacactgaatggtacc

SEQ ID NO: 59 - DNA, 167 bp, synthetic construct
  source 1..167: mol_type = other DNA
gacgacgatatctttgaaaagcactctgtgactctccctgctccctgcggacaaaagcacataatctgctgttacgggtactttcttcacgcgagctttcatgttcagcatgcacgggatcatgcttgtccatgtgaaataaatatggctctctcgtgtccttaaca

SEQ ID NO: 60 - DNA, 192 bp, synthetic construct
  source 1..192: mol_type = other DNA
gaagtgagactgccaggacatgacctgctgtgtggagcccaggagccactggagcgtgtcccatctgtaactcaaaataaagtgcccccaggcatctgcttaagttcttcagggttgttattatgtgggttgatcgacatctccatactgcctgggagagccatcaactgtcattaaagagagttcaagttc

SEQ ID NO: 61 - DNA, 184 bp, synthetic construct
  source 1..184: mol_type = other DNA
ccctcaactgttcactcttagaactttctaaaagcctaaggaaattgctcacattttgacaatgaatactagccaaccatctgttttgcttccctaagaagcatttttttcaacttttattccagtctgagaagcttgtaatttctaaacattgaatgaataaactcaaagaattgccaatgcc

SEQ ID NO: 62 - DNA, 12 bp, synthetic construct
  source 1..12: mol_type = other DNA
aaaaaaaaaaaa

SEQ ID NO: 63 - DNA, 11 bp, synthetic construct
  source 1..11: mol_type = other DNA
aaaaaaaaaaa

(19) United States
(12) Patent Application Publication
(10) Pub. No.: US 2025/0122564 A1
(43) Pub. Date: Apr. 17, 2025

(54) METHODS OF IDENTIFYING ABUNDANCE AND LOCATION OF AN ANALYTE IN A BIOLOGICAL SAMPLE USING SECOND STRAND SYNTHESIS

(71) Applicant: 10x Genomics, Inc., Pleasanton, CA (US)

(72) Inventors: Marco Mignardi, Sundbyberg (SE); Marlon Stoeckius, Stockholm (SE)

(21) Appl. No.: 18/980,743

(22) Filed: Dec. 13, 2024

Related U.S. Application Data
(63) Continuation of application No. 17/368,243, filed on Jul. 6, 2021, now Pat. No. 12,209,280.
(60) Provisional application No. 63/048,584, filed on Jul. 6, 2020.

(51) Int. Cl. C12Q 1/6874

(52) U.S. Cl. CPC C12Q 1/6874

(57) ABSTRACT

Provided herein are methods of identifying abundance and location of an RNA in a biological sample using an adaptor sequence and a primer. Also disclosed herein are kits. compositions, and systems that are used to perform the methods.

### CROSS-REFERENCE TO RELATED APPLICATIONS

This application is a continuation of U.S. patent application Ser. No. 17/368,243, filed on Jul. 6, 2021, which claims priority to U.S. Provisional Patent Application No. 63/048,584, filed on Jul. 6, 2020, the contents of which are incorporated herein by reference in its entirety.

### REFERENCE TO SEQUENCE LISTING

This application contains a Sequence Listing that has been submitted electronically as an XML file named 47706-0220002_SL_ST26.xml. The XML file, created on Dec. 13, 2024, is 60,472 bytes in size. The material in the XML file is hereby incorporated by reference in its entirety.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

RNA sequencing libraries generated from tissue samples can pose some challenges. A targeted approach to insert a sequencing adapter directly to the second-strand DNA which is synthesized on the cDNA previously generated directly on the spatial array would increase efficiency.

### SUMMARY

RNA sequencing libraries generated from formalin-fixed paraffin-embedded tissue samples on spatial arrays are generally short and cDNA could be sequenced directly if it was possible to insert a second sequencing adaptor at the 3′-end of the cDNA. The methods provided herein provide for an efficient, targeted approach for inserting a sequencing adapter directly to the second-strand DNA which is synthesized using the cDNA previously generated directly on the spatial array as a template.

Provided herein are methods of determining abundance and/or location of an RNA molecule in a biological sample. In some instances, the methods include: (a) capturing the RNA molecule from the biological sample on an array comprising a plurality of capture probes, wherein a capture probe of the plurality of capture probes comprises a capture domain and a spatial barcode; (b) extending an end of the capture probe using the RNA molecule as a template, thereby generating an extended capture probe hybridized to the RNA molecule; (c) contacting the extended capture probe with a primer comprising in a 5′ to a 3′ direction: (i) an adapter sequence and (ii) a sequence that specifically binds to the extended capture probe; (d) extending the 3′ end of the primer using the extended capture probe as a template, thereby generating a DNA molecule hybridized to the extended capture probe; and (e) determining (i) all or a part of the sequence of the DNA molecule or a complement thereof, or (ii) all or a part of the sequence of the spatial barcode or a complement thereof, and using the determined sequences of (i) and (ii) to identify the abundance and/or the location of the RNA molecule in the biological sample.

In some instances, the extending in step (b) comprises the use of a reverse transcriptase. In some instances, the methods further include, between steps (b) and (c), digesting the RNA molecule hybridized to the extended capture probe. In some instances, the digesting comprises use of RNAase H or a functional equivalent thereof. In some instances, the extending in step (e) comprises the use of a DNA polymerase.

In some instances, the methods further include releasing the DNA molecule from the extended capture probe, wherein the releasing the DNA molecule comprises heating the DNA molecule to de-hybridize the DNA molecule from the extended capture probe

In some instances, the determining in step (e) comprises sequencing (i) all or a part of the sequence of the RNA molecule or a complement thereof, or (ii) all or a part of the sequence of the spatial barcode or a complement thereof.

In some instances, the adaptor sequence comprises SEQ ID NO:1 (CCTTGGCACACCCGAGAATTCCA). In some instances, the primer sequence comprises a sequence that is complementary to the RNA molecule, or a complement thereof. In some instances, the RNA molecule is an mRNA molecule. In some instances, the capture domain comprises a poly(T) sequence. In some instances, the capture probe further comprises one or more functional domains, a unique molecular identifier, a cleavage domain, and combinations thereof.

In some instances, the capturing in step (a) comprises permeabilizing the biological sample using a permeabilization agent, wherein the permeabilization agent comprises proteinase K or pepsin, thereby releasing the RNA molecule from the biological sample.

In some instances, the biological sample is a tissue section. In some instances, the tissue section is a formalin-fixed paraffin-embedded tissue section. In some instances, the tissue section is a fresh frozen tissue section.

In some instances, the method further comprising imaging the biological sample.

In some instances, the primer is in a primer pool, wherein the primer pool is at a concentration of about 1 μM.

In some instances, the abundance of the RNA molecule is increased by at least about 10% compared to a method that does not utilize the primer.

Also provided herein are methods of identifying a location of an RNA in a biological sample that include: (a) capturing RNA from the biological sample on an array comprising a plurality of capture probes, wherein a capture probe of the plurality of capture probes comprises a capture domain and a spatial barcode; (b) extending an end of the capture probe using the RNA specifically bound by the capture domain as a template, thereby generating an extended capture probe hybridized to the RNA; (c) digesting the RNA hybridized to the extended capture probe; (d) contacting the extended capture probe with a primer comprising in a 5′ to a 3′ direction: (i) an adapter sequence and (ii) a sequence that specifically binds to the extended capture probe; (e) extending the 3′ end of the primer using the extended capture probe as a template, thereby generating a DNA hybridized to the extended capture probe; (f) releasing the generated DNA from the extended capture probe, and (g) determining (i) all or a part of the sequence of the RNA bound by the capture domain or a complement thereof, or (ii) all or a part of the sequence of the spatial barcode or a complement thereof, and using the determined sequences of (i) and (ii) to identify the location of the RNA in the biological sample.

In some embodiments of any of the methods described herein, the extending in step (b) comprises the use of a reverse transcriptase. In some embodiments of any of the methods described herein, the digesting in step (c) comprises the use of RNAase H. In some embodiments of any of the methods described herein, the extending in step (e) comprises the use of a DNA polymerase. In some embodiments of any of the methods described herein, the determining in step (g) comprises sequencing (i) all or a part of the sequence of the RNA or a complement thereof, or (ii) all or a part of the sequence of the spatial barcode or a complement thereof.

In some embodiments of any of the methods described herein, the RNA is an mRNA molecule. In some embodiments of any of the methods described herein, the capture domain comprises a poly(T) sequence. In some embodiments of any of the methods described herein, the capture domain is positioned 3′ relative to the spatial barcode in the capture probe. In some embodiments of any of the methods described herein, the capture probe further comprises a unique molecular identifier. In some embodiments of any of the methods described herein, the capture probe further comprises a cleavage domain. In some embodiments of any of the methods described herein, the capturing in step (a) comprises permeabilizing the biological sample, thereby releasing the RNA from the biological sample.

In some embodiments of any of the methods described herein, the array is a slide. In some embodiments of any of the methods described herein, the slide comprises beads. In some embodiments of any of the methods described herein, the slide comprises wells.

In some embodiments of any of the methods described herein, the biological sample is a tissue sample. In some embodiments of any of the methods described herein, the tissue sample is a tissue section. In some embodiments of any of the methods described herein, the tissue section is a fixed tissue section. In some embodiments of any of the methods described herein, the fixed tissue section is a formalin-fixed paraffin-embedded tissue section. In some embodiments of any of the methods described herein, the tissue section is a fresh, frozen tissue section. Some embodiments of any of the methods described herein further include imaging the biological sample.

Also provided herein are reaction mixtures that include: an array comprising a plurality of capture probes, where a capture probe of the plurality comprises a capture domain that binds specifically to an RNA and a spatial barcode; a reverse transcriptase; RNAse H or a functional equivalent thereof; and a DNA polymerase. In some embodiments of any of the reaction mixtures described herein, the DNA polymerase is DNA polymerase I. Some embodiments of any of the reaction mixtures described herein further include an RNA from a biological sample.

In some embodiments of any of the reaction mixtures described herein, the array is a slide. In some embodiments of any of the reaction mixtures described herein, the slide comprises beads. In some embodiments of any of the reaction mixtures described herein, the slide comprises wells. Some embodiments of any of the reaction mixtures described herein, the reaction mixture further comprises a primer comprising in a 5′ to a 3′ direction: (i) an adapter sequence and (ii) a sequence or a complement thereof present in a 5′ region of the RNA that is specifically bound to the capture domain.

Also provided herein are compositions. In some instances, the compositions include one or more of the following (and any combination thereof): (a) an array comprising a plurality of capture probes, wherein a capture probe of the plurality comprises a capture domain that binds specifically to an RNA and a spatial barcode; (b) a reverse transcriptase enzyme; (c) RNAse H or a functional equivalent thereof; (d) a DNA polymerase; (c) a primer comprising in a 5′ to a 3′ direction: (i) an adapter sequence and (ii) a sequence or a complement thereof present in a 5′ region of the RNA molecule that is specifically bound to the capture domain; and (f) an RNA molecule from a biological sample.

Also provided herein are kits. In some instances, the kits include one or more of the following (and any combination thereof): (a) an array comprising a plurality of capture probes, wherein a capture probe of the plurality comprises a capture domain that binds specifically to an RNA and a spatial barcode; (b) a reverse transcriptase; (c) RNAse H or a functional equivalent thereof; (d) a DNA polymerase; (c) a primer comprising in a 5′ to a 3′ direction: (i) an adapter and (ii) a sequence or a complement thereof present in a 5′ region of the RNA molecule that is specifically bound to the capture domain; and (f) instructions for performing any of the methods described herein.

In some instances, the kits include: an array comprising a plurality of capture probes, where a capture probe of the plurality comprises a capture domain that binds specifically to an RNA and a spatial barcode; a reverse transcriptase; RNAse H or a functional equivalent thereof; and a DNA polymerase. In some embodiments of any of the kits described herein, the DNA polymerase is DNA polymerase I. In some embodiments of any of the kits described herein, the capture domain is positioned 3′ of the spatial barcode in the capture domain. In some embodiments of any of the kits described herein, the capture probe further comprises a unique molecular identifier. In some embodiments of any of the kits described herein, the capture probe further comprises a cleavage domain. Some embodiments of any of the kits described herein further include an RNA from a biological sample.

In some embodiments of any of the kits described herein, the array is a slide. In some embodiments of any of the kits described herein, the slide comprises beads. In some embodiments of any of the kits described herein, the slide comprises wells. Some embodiments of any of the kits described herein further include a primer comprising in a 5′ to a 3′ direction: (i) an adapter and (ii) a sequence or a complement thereof present in a 5′ region of the RNA that is specifically bound to the capture domain.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A-6C are schematics illustrating how streptavidin cell tags can be utilized in an array-based system to produce spatially-barcoded cells or cellular contents.

FIGS. 7A and 7B show a schematic illustrating an exemplary embodiment of the methods described herein. smRNA R2: R2 primer handle; UMI: unique molecular identifier; Spat Barcode: spatial barcode; TruSeq™ R1:R1 primer handle (e.g., RNA-seq library preparation primers).

FIGS. 8A and 8B show electrophoresis fragment sizes and library traces of Groups A-D.

FIG. 9 shows unique molecular identifiers (UMIs) for 20 targeted genes in a comparison of genome-wide expression results compared to targeted second strand results.

FIGS. 10A and 10B show electrophoresis fragment sizes and library traces of using hot-start amplification mix with and without template switching oligonucleotides.

FIG. 11 shows UMIs for targeted genes using second strand synthesis.

FIGS. 12A and 12B show library traces of using hot-start amplification mix with and without template switching oligonucleotides while increasing pre-second-strand synthesis and second-strand synthesis temperatures.

FIGS. 13A-13E shows UMIs for whole genome detection versus second strand synthesis of low, medium, and negative control analytes. FIG. 13D shows an enlarged version of the dotted subset of FIG. 13A. FIG. 13E shows an enlarged version of the dotted subset of FIG. 13B.

FIG. 14 shows UMIs in a comparison of genome-wide expression results compared to targeted second strand synthesis (TSS) results.

FIG. 15 shows UMIs in a comparison of target analytes versus non-target analytes when temperature is varied using hot-start amplification mix.

FIG. 16 shows spatial analysis of target genes (Tnnt1, Prkcd, Nr4a2, Hs3st2, and Cldn11) using whole genome detection methods versus targeted second strand synthesis (TSS) methods.

### DETAILED DESCRIPTION

### I. Introduction

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363 (6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), both of which are available at the 10× Genomics Support Documentation website, and can be used herein in any combination. Further non-limiting aspects of spatial analysis methodologies and compositions are described herein.

Some general terminologies that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A biological sample is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

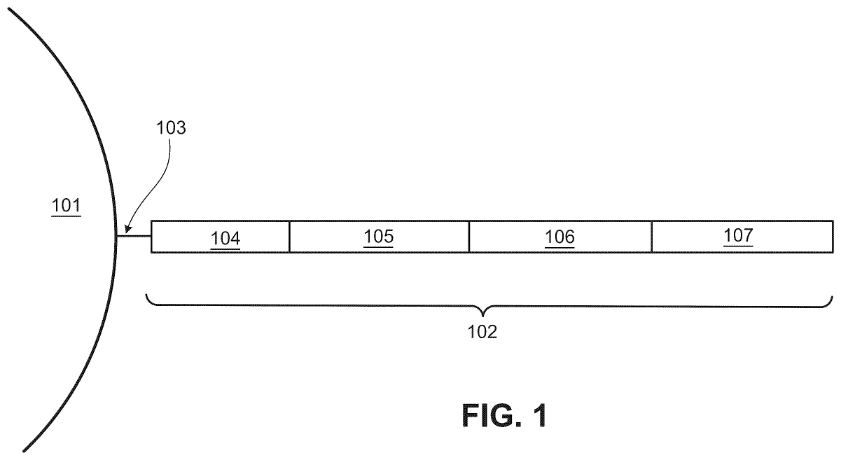

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent™ Proton or PGM (i.e., ion semiconductor sequencing), Illumina™ sequencing instruments (e.g., sequencing by synthesis), PacBio™ (e.g., HiFi sequencing), OXFORD NANOPORE, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent™ Proton or PGM sequencing (i.e., ion semiconductor sequencing), Illumina™ sequencing (e.g., sequencing by synthesis), PacBio™ SMRT™ sequencing (e.g., HiFi sequencing), and OXFORD NANOPORE sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

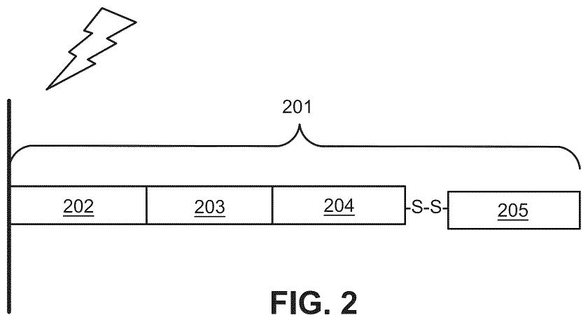

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

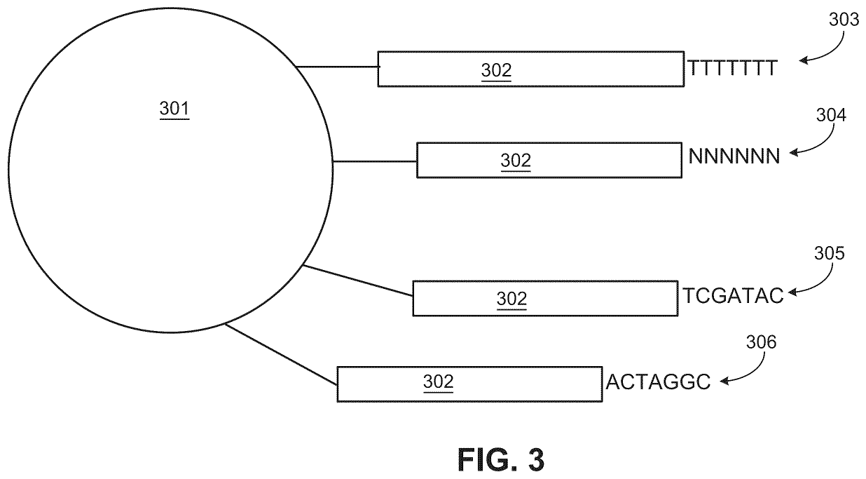

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly (T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

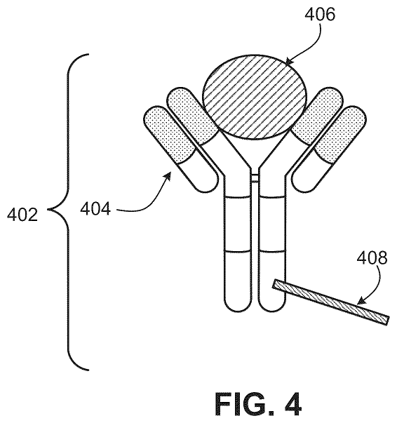

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

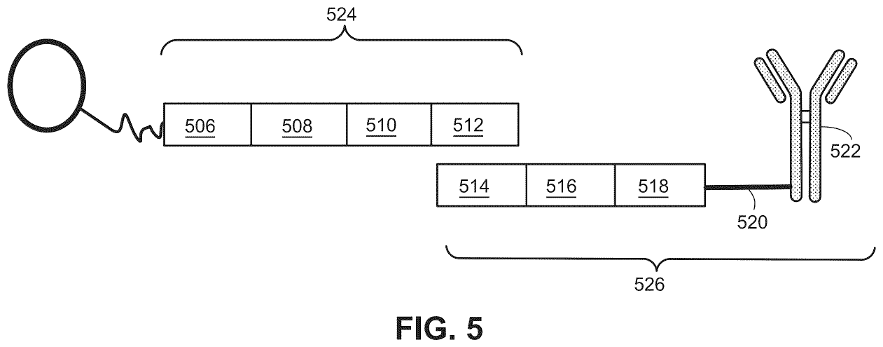

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain to couple to the analyte binding moiety 522.

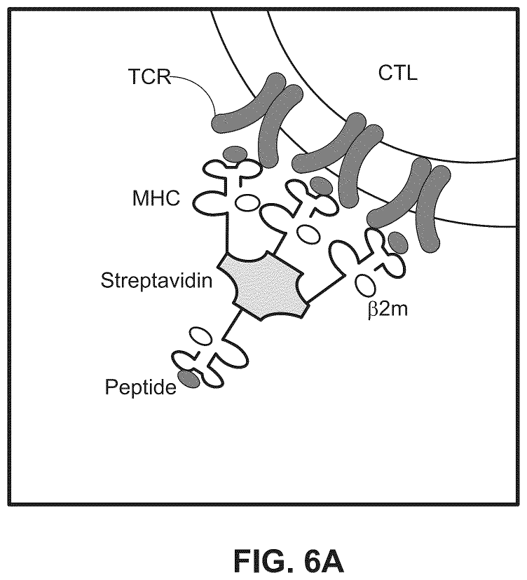

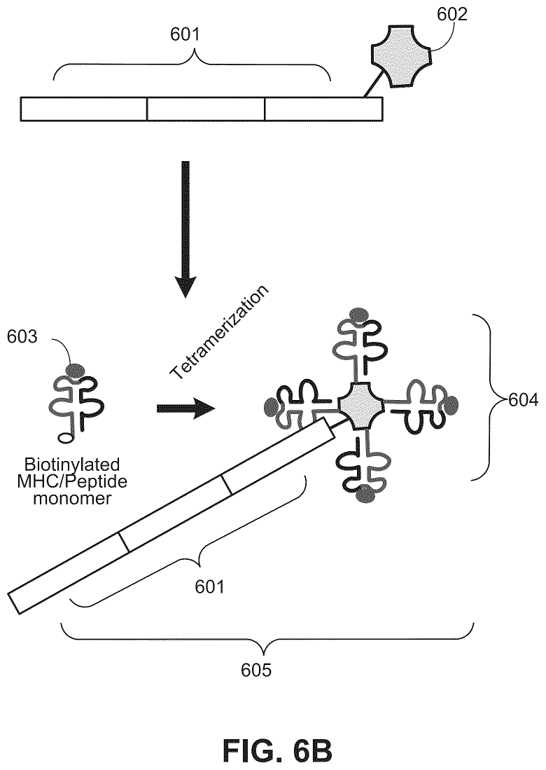

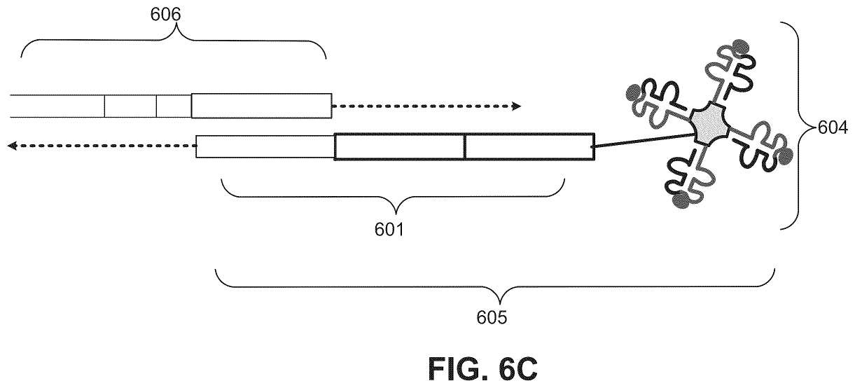

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MHC/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601, thereby forming a tetramerized MHC complex 604. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of the corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a nucleic acid molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.
Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(c) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev D, dated October 2020), and/or the Visium Spatial Tissue Optimization Reagent Kits User Guide (e.g., Rev D, dated October 2020). In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Methods and Compositions for Analyte Detection in a Biological Sample

### A. Introduction

RNA sequencing libraries generated from formalin-fixed paraffin-embedded (FFPE) tissue samples on spatial arrays are generally short and cDNA could be sequenced directly if it was possible to insert a second sequencing adaptor at the 3′-end of the cDNA. The methods provided herein provide for an efficient, targeted approach for inserting a sequencing adapter directly to the second-strand DNA which is synthesized using the cDNA previously generated directly on the spatial array as a template. However, the methods are not limited to FFPE tissues as the methods are equally amenable with other tissue types, such as fresh frozen samples or alternatively fixed samples (e.g., methanol, acetone, etc.). Thus, in some instances, the biological sample is taken from a sample fixed with formalin (e.g., an FFPE sample). In other instances, the biological sample is not fixed, and can be a freshly-obtained sample or a frozen sample.

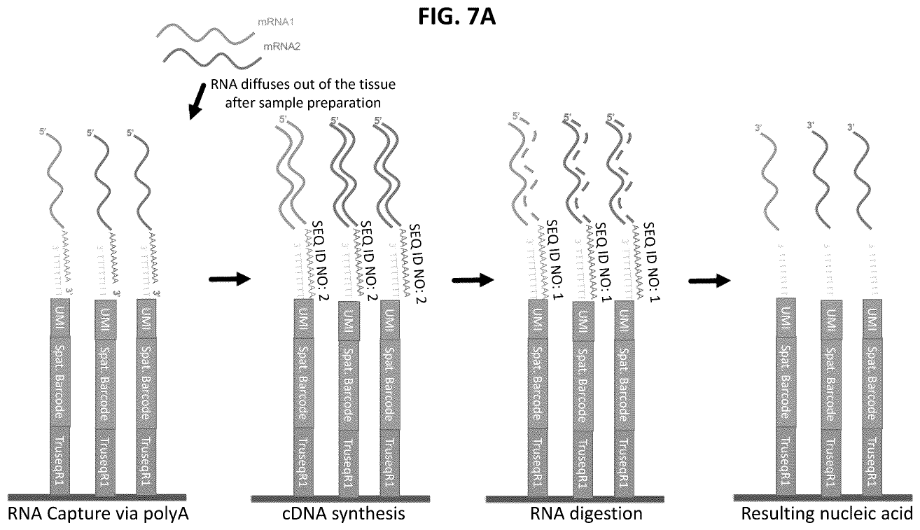

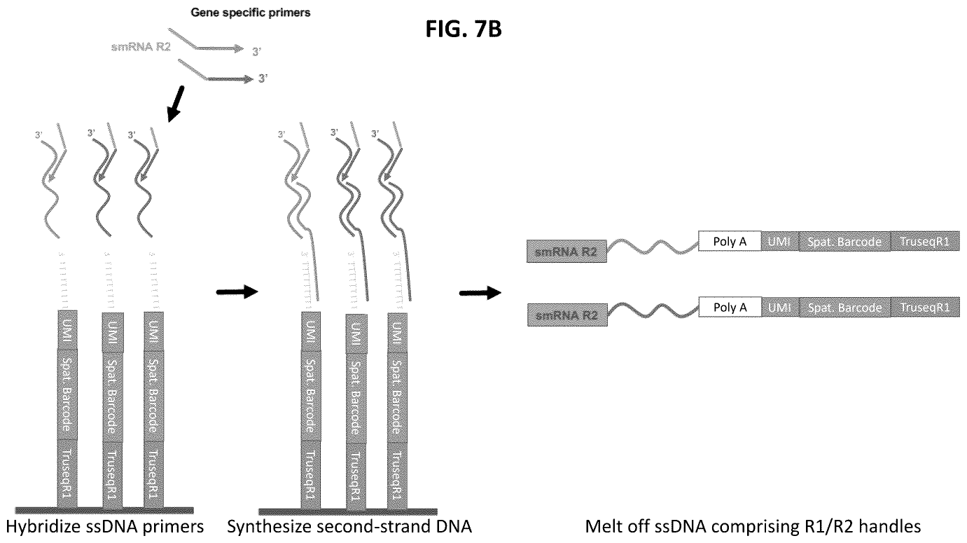

In some workflows of spatial analyses, gene-specific primers containing a universal sequence are utilized in a targeted approach for second strand synthesis. An exemplary embodiment of the methods on FFPE tissue described herein is depicted in FIGS. 7A-7B. It is appreciated that the workflow would be similar on fresh frozen tissue. FIGS. 7A-7B depicts mRNA capture from a FFPE tissue sample (not shown) that is placed on a spatial array after permeabilizing the FFPE tissue sample to release the mRNA (e.g., mRNA1; mRNA2 as shown in FIG. 7A). After permeabilization, mRNA molecules are captured by capture probes on the spatial array (e.g., by hybridization of the poly(A) tail of the analyte to the poly(T) sequence of the capture probe). As shown in FIG. 7A, The capture probe sequence comprises a first sequencing adaptor proximal to the substrate, a spatial barcode, optionally a unique molecule identifier (UMI) sequence, and a capture domain, in this example the capture domain comprises a polyT sequence for mRNA capture. Capture domain(s) can be extended to generate first strand cDNA molecule(s) using the mRNA bound to capture domain(s) as a template. After cDNA synthesis, the RNA that was used as a template for cDNA synthesis is digested by RNase treatment. The extended capture probe(s) is/are single-stranded cDNA molecule(s), which is/are still attached to the spatial array. The extended capture probe(s) is/are then contacted with primer(s) comprising a second adapter sequence (e.g., a sequencing adapter sequence) and a sequence that specifically binds to the extended capture probe. The primer(s) is/are then extended using the extended capture probe(s) as a template, thereby generating a DNA(s) hybridized to the extended capture probe(s) (also called the “second strand(s)”). The second strand(s) can then be recovered and used to prepare libraries for subsequent processing and analysis (e.g., sequencing using any of the methods described herein, e.g., high throughput sequencing, e.g., Illumina™ sequencing (e.g., sequencing by synthesis)).

Provided herein are methods of identifying a location of an RNA in a biological sample that include: (a) contacting a biological sample (e.g., any of the exemplary biological samples described herein) with an array comprising a plurality of capture probes, wherein a capture probe of the plurality of capture probes comprises a capture domain (e.g., any of the exemplary capture domains described herein), a spatial barcode and a first adaptor sequence (e.g., a first sequencing primer sequence); (b) extending an end of the capture probe using the captured RNA (e.g., any of the exemplary types of RNA described herein, e.g., mRNA) specifically bound by the capture domain as a template, thereby generating an extended capture probe hybridized to the RNA; (c) digesting the RNA hybridized to the extended capture probe; (d) contacting the extended capture probe with a primer comprising in a 5′ to a 3′ direction: (i) an adapter sequence (e.g., a second sequencing adapter sequence, e.g., a universal sequencing adapter sequence) and (ii) a sequence that specifically binds to (e.g., at least a portion of) the extended capture probe; (e) extending the 3′ end of the primer using the extended capture probe as a template, thereby generating a DNA hybridized to the extended capture probe; (f) releasing the generated DNA from the extended capture probe; and (g) determining (i) all or a part of the sequence of the RNA bound by the capture domain or a complement thereof, or (ii) all or a part of the sequence of the spatial barcode or a complement thereof, and using the determined sequences of (i) and (ii) to identify the location of the RNA in the biological sample.

### B. Methods for Analyte Capture and Capture Probe Extension

In some instances, after preparing the biological sample for spatial analysis, the analyte (e.g., mRNA) is captured by a capture probe on an array. In some embodiments of any of the methods described herein, the capture domain comprises a poly(T) sequence. In some instances, the analyte hybridizes to the poly(T) sequence. In some embodiments, the capture domain does not comprise a poly(T) sequence. In some embodiments, the capture domain comprises a sequence that is substantially complementary to a contiguous sequence present in the RNA. The capture domain can be about 5 to about 40 nucleotides (e.g., about 5 to about 35 nucleotides, about 5 to about 30 nucleotides, about 5 to about 25 nucleotides, about 5 to about 20 nucleotides, about 5 to about 15 nucleotides, about 5 to about 10 nucleotides, about 10 to about 40 nucleotides, about 10 to about 35 nucleotides, about 10 to about 30 nucleotides, about 10 to about 25 nucleotides, about 10 to about 20 nucleotides, about 10 to about 15 nucleotides, about 15 to about 40 nucleotides, about 15 to about 35 nucleotides, about 15 to about 30 nucleotides, about 15 to about 25 nucleotides, about 15 to about 20 nucleotides, about 20 to about 40 nucleotides, about 20 to about 35 nucleotides, about 20 to about 30 nucleotides, about 20 to about 25 nucleotides, about 25 to about 40 nucleotides, about 25 to about 35 nucleotides, about 25 to about 30 nucleotides, about 30 to about 40 nucleotides, about 30 to about 35 nucleotides, or about 35 to about 40 nucleotides) in length. In some instances, one or more capture probes on the spatial array further include a spatial barcode and/or a unique molecular identifier (UMI).

In some embodiments of any of the methods described herein, the plurality of capture probes are affixed (i.e., attached) to an array. In some embodiments of any of the methods described herein, the array is a slide (e.g., a slide comprising beads or a slide comprising wells (e.g., microwells)). An array can also have one or more of any of the exemplary characteristics of arrays described herein.

In some embodiments, the capture domain is positioned 3′ relative to the spatial barcode in the capture probe. In some embodiments of any of the methods provided herein, the capture probe further includes a unique molecular identifier, a cleavage domain (e.g., any of the exemplary cleavage domains described herein), or both.

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, after analyte capture, the capture probe can be extended (an “extended capture probe,” e.g., as described herein). In some embodiments, the capture probe is extended at the 3′ end. For example, extending a capture probe can include generating cDNA from a captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., reverse transcription, step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, the capture probe is extended using fluorescently labeled nucleotides. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some embodiments, digesting the RNA from the RNA:DNA hybrid comprises the use of an RNase that digests RNA from a RNA:DNA hybrid, for example, RNAse H or a functional equivalent thereof.

### C. Methods and Compositions for Second Strand Synthesis

After extension of the capture probe and degradation of the analyte, target-specific primers are added to the sample. In some instances, a target-specific primer as described herein comprises a sequence that is complementary to the extended capture probe. In some instances, a target-specific primer comprises a sequence that is complementary to the extended capture probe at the sequence complementary to the analyte. Thus, in some instances, the primer includes a sequence that is specific for one or more targets of interest.

The sequence in the primer that specifically binds to (e.g., at least a portion of) the extended capture probe can about 15 to about 50 nucleotides (e.g., about 15 to about 45 nucleotides, about 15 to about 40 nucleotides, about 15 to about 35 nucleotides, about 15 to about 30 nucleotides, about 15 to about 25 nucleotides, about 15 to about 20 nucleotides, about 20 to about 50 nucleotides, about 20 to about 45 nucleotides, about 20 to about 40 nucleotides, about 20 to about 35 nucleotides, about 20 to about 30 nucleotides, about 20 to about 25 nucleotides, about 25 to about 50 nucleotides, about 25 to about 45 nucleotides, about 25 to about 40 nucleotides, about 25 to about 35 nucleotides, about 25 to about 30 nucleotides, about 30 to about 50 nucleotides, about 30 to about 45 nucleotides, about 30 to about 40 nucleotides, about 30 to about 35 nucleotides, about 35 to about 50 nucleotides, about 35 to about 45 nucleotides, about 35 to about 40 nucleotides, about 40 to about 50 nucleotides, about 40 to about 45 nucleotides, or about 45 to about 50 nucleotides) long. In some embodiments, the sequence in the primer that specifically binds to the extended capture probe comprises a sequence corresponding to a contiguous sequence present in the RNA that is specifically bound to the capture domain. For example, the sequence in the primer that specifically binds to the extended capture probe corresponds to a contiguous sequence in the RNA (that is specifically bound to the capture domain) that is about 20 to about 1,000 nucleotides (e.g., about 20 to about 1000 nucleotides, about 20 to about 900 nucleotides, about 20 to about 800 nucleotides, about 20 to about 700 nucleotides, about 20 to about 600 nucleotides, about 20 to about 500 nucleotides, about 20 to about 400 nucleotides, about 20 to about 300 nucleotides, about 20 to about 200 nucleotides, about 20 to about 150 nucleotides, about 20 to about 100 nucleotides, about 20 to about 80 nucleotides, about 20 to about 60 nucleotides, about 20 to about 40 nucleotides,) 5′ to the 3′ end of the RNA that is specifically bound to the capture domain.

Primers (and groups of primers) can be designed to be specific to only a few analytes (e.g., about 2 analytes to about 20 analytes) or more. The specificity of primers depends on the design of the sequence that hybridizes to the extended capture probe. In some instances, primers can be designed to target about 100 analytes, about 500 analytes, about 1000 analytes, and even the entire genome.

In some instances, at the 5′ end, the primer further includes an adaptor sequence. In some instances, the adapter sequence in the primer can include a sequencing adapter sequence (e.g., an adapter sequence that can be used to perform sequencing using any of the exemplary sequencing methods described herein). In some embodiments, the adapter sequence can be an Illumina™ sequencing adapter sequence (e.g., via sequencing by synthesis). In some embodiments, the adapter sequence can be about 15 to about 45 nucleotides (e.g., about 15 to about 45 nucleotides, about 15 to about 40 nucleotides, about 15 to about 35 nucleotides, about 15 to about 30 nucleotides, about 15 to about 25 nucleotides, or about 15 to about 20 nucleotides, about 20 to about 45 nucleotides, about 20 to about 40 nucleotides, about 20 to about 35 nucleotides, about 20 to about 30 nucleotides, about 20 to about 25 nucleotides, about 25 to about 45 nucleotides, about 25 to about 40 nucleotides, about 25 to about 35 nucleotides, about 25 to about 30 nucleotides, about 30 to about 45 nucleotides, about 30 to about 40 nucleotides, about 30 to about 35 nucleotides, about 35 to about 45 nucleotides, about 35 to about 40 nucleotides, or about 40 to about 45 nucleotides) long. In some embodiments, the adapter sequence comprises a sequence of CCTTGGCACACCCGAGAATTCCA (SEQ ID NO:1). In some embodiments, the adapter sequence can be a universal sequence.

In some embodiments, the step of extending the 3′ end of the primer using the extended capture probe as a template, thereby generating a DNA hybridized to the extended capture probe, includes the use of a DNA polymerase, e.g., DNA polymerase I or any of the other exemplary DNA polymerases described herein or known in the art.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, WI). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9°N) DNA ligase (9°N™ DNA ligase, New England Biolabs), Ampligase™ (available from Lucigen, Middleton, WI), and SplintR (available from New England Biolabs, Ipswich, MA). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments of any of the methods described herein, the releasing of the generated DNA from the extended capture probe can be performed using heat and/or a solution (e.g., a solution having an increased salt concentration).

After release of the generated DNA molecule, the resulting generated DNA molecule—as shown in FIG. 7B—is single stranded and includes, without limitation, at least a primer adaptor (e.g. read 2 sequencing handle), a sequence complementary to the extended capture probe, a polyA sequence, a unique molecular identifier (UMI), a spatial barcode, a second primer sequence (e.g. read 1 sequencing handle), or some combination thereof.

In some instances, the methods of generating a DNA molecule from the extended capture probe comprises one or more steps of heating the samples. In some instances, the heating step is performed prior to second strand synthesis. In some instances, the heating step performed prior to second strand synthesis is performed at about 98° C. In some instances, the heating step performed prior to second strand synthesis is performed from about 80° C. to about 100° C. (e.g., about 80, 81, 82, 83, 84, 85, 86, 87, 88, 89, 90, 91, 92, 93, 94, 95, 96, 97, 98, 99, or 100° C.). In some instances, the heating step is performed during second strand synthesis. In some instances, the temperature of the heating step during second strand synthesis is about 65° C. and can range from 50° C. to 80° C. (e.g., 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C.). In some instance, any of the heating steps can be performed from 1 to 30 minutes (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes). In some instances, the buffer for second strand synthesis a Hot Start Master Mix (e.g., a Hot Start Taq 2X Master Mix; e.g., New England Biolabs®, Inc.

In some embodiments, the methods further include a determining (e.g., sequencing) step. In some instances, the determining step comprises determining the sequence of (i) all or a part of the sequence of generated DNA or a complement thereof, or (ii) all or a part of the sequence of the spatial barcode or a complement thereof. In some embodiments, the sequencing can be performed using any of the exemplary sequencing methods described herein (e.g., high throughput sequencing). In some instance, the generated DNA (e.g., the second strand molecule) can be amplified via PCR prior to library construction. The generated DNA can then be enzymatically fragmented and size-selected in order to optimize for amplicon size. P5 and P7 sequences directed to capturing the amplicons on a sequencing flowcell (Illumina™ sequencing instruments (e.g., sequencing by synthesis)) can be appended to the amplicons, i7, and i5 can be used as sample indexes, and TruSeq™ Read 2 (e.g., an RNA-seq library preparation primer) can be added via End Repair, A-tailing, Adaptor Ligation, and PCR. The cDNA fragments can then be sequenced using paired-end sequencing using TruSeq™ Read 1 and TruSeq™ Read 2 (e.g., an RNA-seq library preparation primers) as sequencing primer sites. The additional sequences are directed toward Illumina™ sequencing instruments (e.g., sequencing by synthesis) or sequencing instruments that utilize those sequences; however a skilled artisan will understand that additional or alternative sequences used by other sequencing instruments or technologies are also equally applicable for use in the aforementioned methods.

A wide variety of different sequencing methods can be used to analyze barcoded analyte. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

### D. Biological Samples and Analytes

Methods disclosed herein can be performed on any type of sample (also interchangeably called “biological sample”). In some embodiments, the sample is a fresh tissue. In some embodiments, the sample is a frozen sample. In some embodiments, the sample was previously frozen. In some embodiments, the sample is a formalin-fixed, paraffin embedded (FFPE) sample.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells,

In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E will allow for morphological context overlaid with transcriptomic analysis. However, depending on the need some samples may be stained with only a nuclear stain, such as staining a sample with only hematoxylin and not eosin, when location of a cell nucleus is needed.

In some embodiments, a biological sample (e.g. tissue section) can be fixed with methanol, stained with hematoxylin and eosin, and imaged. In some embodiments, fixing, staining, and imaging occurs before one or more probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the FFPE sample is deparaffinized, permeabilized, equilibrated, and blocked before target probe oligonucleotides are added. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

The biological samples included herein comprise one or more analytes. Analytes can be broadly classified into one of two groups: nucleic acid analytes, and non-nucleic acid analytes.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte can be an organelle (e.g., nuclei or mitochondria).

Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

Additional examples of analytes are disclosed in WO 2020/176788 and/or U.S. Patent Application Publication No. 2020/0277663, each of which is incorporated by reference in its entirety.

### E. Methods for Preparing a Biological Sample for Spatial Analysis

### (i) Imaging and Staining

Prior to analyte migration and capture, in some instances, biological samples can be stained using a wide variety of stains and staining techniques. In some instances, the biological sample is a section on a slide (e.g., a 10 μm section). In some instances, the biological sample is dried after placement onto a glass slide. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3X SSC, 2% BSA, 0.1% Triton X, 1 U/μl RNAse inhibitor for 10 minutes at 4° C.) before being stained with fluorescent primary antibodies (1:100 in 3XSSC, 2% BSA, 0.1% Triton X, 1 U/μl RNAse inhibitor for 30 minutes at 4° C.). The biological sample can be washed, coverslipped (in glycerol +1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

### (ii) Preparation of Sample for Analyte Migration and Capture

In some instances, the biological sample is deparaffinized. Deparaffinization can be achieved using any method known in the art. For example, in some instances, the biological samples is treated with a series of washes that include xylene and various concentrations of ethanol. In some instances, methods of deparaffinization include treatment of xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° C. Just prior to decrosslinking, the biological sample can be treated with an acid (e.g., 0.1M HCl for about 1 minute). After the decrosslinking step, the biological sample can be washed (e.g., with 1× PBST).

In some instances, the methods of preparing a biological sample for analyte capture include permeabilizing the sample. In some instances, the biological sample is permeabilized using a phosphate buffer. In some instances, the phosphate buffer is PBS (e.g., 1× PBS). In some instances, the phosphate buffer is PBST (e.g., 1× PBST). In some instances, the permeabilization step is performed multiple times (e.g., 3 times at 5 minutes each).

In some instances, the methods of preparing a biological sample for analyte capture include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA such as from brewer's yeast (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

### III. Reaction Mixtures and Kits

Also provided herein are reaction mixtures that include: an array comprising a plurality of capture probes, where a capture probe of the plurality comprises a capture domain (e.g., any of the exemplary capture domains described herein or known in the art) that binds specifically to an RNA (e.g., any of the exemplary types of RNA described herein or known in the art) and a spatial barcode; a reverse transcriptase (e.g., any of the exemplary reverse transcriptases described herein or known in the art); RNAse H or a functional equivalent thereof; and a DNA polymerase (e.g., any of the exemplary DNA polymerases described herein or known in the art).

Also provided herein are kits that include: an array comprising a plurality of capture probes, where a capture probe of the plurality comprises a capture domain (e.g., any of the exemplary capture domains described herein or known in the art) that binds specifically to an RNA (e.g., any of the exemplary types of RNA described herein or known in the art) and a spatial barcode; a reverse transcriptase (e.g., any of the exemplary reverse transcriptases described herein or known in the art); RNAse H or a functional equivalent thereof; and a DNA polymerase (e.g., any of the exemplary DNA polymerases described herein or known in the art).

In some embodiments of any of the reaction mixtures or kits described herein, the capture domain can be any of the capture domains described herein. In some embodiments, the capture domain can comprise a poly(T) sequence. In some embodiments, the capture domain does not comprise a poly(T) sequence. In some embodiments, the capture domain comprises a sequence that is substantially complementary to a contiguous sequence present in the RNA. The capture domain can be about 5 to about 40 nucleotides (e.g., or any of the subranges of this range described herein) in length.

In some embodiments, the capture domain is positioned 3′ relative to the spatial barcode in the capture probe. In some embodiments of any of the reaction mixtures or kits provided herein, the capture probe further includes a unique molecular identifier, a cleavage domain (e.g., any of the exemplary cleavage domains described herein), or both.

In some embodiments of any of the reaction mixtures or kits described herein, the plurality of capture probes are affixed (i.e., attached) to an array. In some embodiments of any of the reaction mixtures or kits described herein, the array is a slide (e.g., a slide comprising beads or a slide comprising wells (e.g., microwells)). An array can also have one or more of any of the exemplary characteristics of arrays described herein.

Some embodiments of any of the reaction mixtures or kits described herein can further include a primer comprising in a 5′ to a 3′ direction: (i) an adapter sequence (e.g., any of the exemplary adapter sequences described herein) and (ii) a sequence or a complement thereof present in a 5′ region of the RNA that is specifically bound to the capture domain.

The sequence or complement thereof present in a 5′ region of the RNA that is specifically bound to the capture domain can be about 15 to about 50 nucleotides (e.g., or any of the subranges of this range described herein) long. In some embodiments, the sequence present in the 5′ region of the RNA (that is specifically bound to the capture domain) is about 20 to about 1,000 nucleotides (e.g., or any of the subranges of this range described herein) 5′ to the 3′ end of the RNA that is specifically bound to the capture domain.

Some embodiments of the kits described herein further include a solution that can be used to dissociate two strands of DNA (e.g., an extended capture probe and a DNA that is hybridized to the extended capture probe). In some embodiments, the solution that can be used to dissociate two strands of DNA can have an increased salt concentration.

In some embodiments of any of the reaction mixtures described herein, the reaction mixture can include an RNA from a biological sample (e.g., an mRNA or any of the other types of RNA described herein or known in the art).

In some embodiments of any of the kits or reaction mixtures described herein, the kit or reaction mixture can further include one or more permeabilization reagents (e.g., one or more of any of the permeabilization reagents described herein).

Some embodiments of any of the kits described herein can further include a staining agent. In some embodiments, a staining agent can include an optical label, e.g., a fluorescent, a radioactive, a chemiluminescent, a calorimetric, or a colorimetric detectable label. In some embodiments, a staining agent can be a fluorescent antibody directed to a target analyte (e.g., cell surface or intracellular proteins). In some embodiments, a staining agent can be a chemical stain, such as hematoxylin and eosin (H&E) or periodic acid-schiff (PAS).

Some embodiments of any of the kits described herein can further include instructions for performing any of the methods described herein.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1: Adding an Adapter to a Second Strand Complementary to an Extended Capture Probe

An experiment was performed to demonstrate the performance of the methods described herein in determining the location of 20 exemplary RNA molecules in FFPE mouse brain tissue.

Briefly, a FFPE mouse brain tissue section was placed on a spatial array comprising a plurality of capture probes. The tissue section was permeabilized to release mRNA from the sample. After permeabilization, mRNA molecules were captured by capture probes via hybridization of the poly(A) tail of the mRNA to the poly(T) sequence of the capture probe. The capture probe was extended using a polymerase to generate a first strand cDNA molecule, using the mRNA bound to capture domain as a template. After cDNA synthesis, the RNA that was used as a template for first strand cDNA synthesis was digested by RNase H, leaving a single-stranded extended capture probe. The extended capture probes were contacted with primers comprising an adapter sequence (e.g., a second sequencing adapter sequence; e.g., SEQ ID NO:1 (CCTTGGCACACCCGAGAATTCCA)) and a sequence that specifically binds to the extended capture probe (e.g., binding to the target sequences shown in Table 2). The primers were extended using the extended capture probe as a template, thereby generating a DNA molecule that is hybridized to the extended capture probe and that is termed the second strand. The second strands were recovered and were used to prepare libraries for subsequent processing and analysis (e.g., sequencing using any of the methods described herein, e.g., high throughput sequencing, e.g., Illumina™ sequencing (e.g., sequencing by synthesis)).

Tables 1 and 2 show the list of the 20 exemplary RNA sequences and the sequences that specifically binds to the extended capture probe (“target sequence;” SEQ ID NOs: 2-21) using a primer sequence (e.g., one of SEQ ID NOs: 22-41) in Table 2. The captured sequences for each analyte are shown in Table 3 (SEQ ID NOs: 42-61).

All primers include sequences that correspond to sequences in the 3′ UTR of the target RNAs, except two primers which include a sequence that spans an exon and the 3′ UTR of the target RNA. The four groups of genes shown in Table 1 are based on varying levels of analyte expression and UMI detection, with Group 1 having the highest expression and abundance of detection. The primer sequences were blasted and checked for self-dimer and cross-primer dimers. The data in Table 1 demonstrate the ability of these methods to add a sequencing adapter to a 5′ end of a DNA that is complementary to the extended capture probe (“the second strand”), and the subsequent successful sequencing of the second strand. The 20 exemplary RNAs listed in Table 1 include mRNAs for the housekeeping genes of GAPDH, ACTB, B2M, and FGB.

TABLE 1
Targeted Analytes

| | | | | | | | nt to | Primers |
|---|---|---|---|---|---|---|---|---|
| | | | | | | | poly | on 3′ |
| Analyte | Group | num_umis | num_barcodes_cells | num_reads_cells | num_umis_cells | Genomic location | A | UTR |
| Mbp | 1 | 405505 | 2858 | 1254340 | 381413 | 18:82558495-82558519 | 187 | yes |
| Gapdh | 1 | 395061 | 2858 | 962940 | 353511 | 6:125162022- | 171 | spanning |
| | | | | | | 125162044 | | |
| Plp1 | 1 | 275818 | 2854 | 768966 | 262333 | X:136839533- | 181 | yes |
| | | | | | | 136839556 | | |
| Actb | 1 | 190193 | 2855 | 390405 | 170617 | 5:142903261- | 146 | yes |
| | | | | | | 142903285 | | |
| Itm2c | 1 | 105108 | 2856 | 271887 | 95619 | 1:85908500-85908524 | 154 | yes |
| Vsnl1 | 1 | 91104 | 2841 | 234144 | 83811 | 12:11325402-11325425 | 161 | yes |
| B2m | 2 | 38662 | 2849 | 97390 | 34986 | 2:122152880- | 179 | yes |
| | | | | | | 122152904 | | |
| Plekhb1 | 2 | 34608 | 2800 | 102306 | 32596 | 7:100643054- | 162 | yes |
| | | | | | | 100643078 | | |
| Cldn11 | 2 | 27050 | 2561 | 59944 | 25186 | 3:31164143-31164167 | 160 | yes |
| Ahi1 | 2 | 20613 | 2709 | 54378 | 18983 | 10:21080222- | 188 | yes |
| | | | | | | 21080244 | | |
| Pde1b | 2 | 14315 | 2582 | 40455 | 13030 | 15:103529881- | 149 | yes |
| | | | | | | 103529905 | | |
| Adarb1 | 2 | 12245 | 2562 | 31584 | 11302 | 10:77290903-77290927 | 178 | yes |
| Zcchc12 | 3 | 9886 | 2299 | 27061 | 8929 | X:36198962-36198986 | 174 | yes |
| Penk | 3 | 6384 | 1735 | 16410 | 5645 | 4:4133699-4133723 | 171 | yes |
| Nr4a2 | 3 | 4663 | 1571 | 12100 | 4389 | 2:57106954-57106978 | 126 | yes |
| Gpr88 | 3 | 3090 | 980 | 8195 | 2756 | 3:116249856- | 205 | yes |
| | | | | | | 116249881 | | |
| Prkcd | 3 | 3086 | 1018 | 5601 | 2931 | 14:30595537-30595561 | 184 | yes |
| Hs3st2 | 3 | 2825 | 1094 | 7316 | 2573 | 7:121501604- | 144 | yes |
| | | | | | | 121501628 | | |
| Tnnt1 | 4 | 1173 | 521 | 2601 | 1108 | 7:4504739-4504761 | 172 | spanning |
| Fgb | 4 | 0 | 0 | 0 | 0 | 3:83040300-83040324 | 162 | yes |

TABLE 2
Target Sequences and Primers

| | | Target | | Primer | |
|---|---|---|---|---|---|
| Ensembl | Target | Sequence | | Sequence | |
| Identifier | Analyte | Identifier | Target Sequence | Identifier | Primer Sequence |
| ENSMUSG00000041607 | Mbp | 2 | ACTGACACTGGAA | 22 | CCTTGGCACCCGAGAATTCCAACTG |
| | | | TAGGAATGTGAT | | ACACTGGAATAGGAATGTGAT |
| ENSMUSG00000057666 | Gapdh | 3 | CTCCAAGGAGTAA | 23 | CCTTGGCACCCGAGAATTCCACTCC |
| | | | GAAACCCTGG | | AAGGAGTAAGAAACCCTGG |
| ENSMUSG00000031425 | Plp1 | 4 | CTGCCCTCTGGGA | 24 | CCTTGGCACCCGAGAATTCCACTGC |
| | | | TGGATCTATAG | | CCTCTGGGATGGATCTATAG |
| ENSMUSG00000029580 | Actb | 5 | TTTTAATTTCTGAA | 25 | CCTTGGCACCCGAGAATTCCATTTT |
| | | | TGGCCCAGGTC | | AATTTCTGAATGGCCCAGGTC |
| ENSMUSG00000026223 | Itm2c | 6 | TCCTGCATGTTTTT | 26 | CCTTGGCACCCGAGAATTCCATCCT |
| | | | ACTGATGTTCG | | GCATGTTTTTACTGATGTTCG |
| ENSMUSG00000054459 | Vsnl1 | 7 | CTGGAATTTGCAG | 27 | CCTTGGCACCCGAGAATTCCACTGG |
| | | | AATGACTGGAAG | | AATTTGCAGAATGACTGGAAG |
| ENSMUSG00000060802 | B2m | 8 | ATCATATGCCAAA | 28 | CCTTGGCACCCGAGAATTCCAATCA |
| | | | CCCTCTGTACTT | | TATGCCAAACCCTCTGTACTT |
| ENSMUSG00000030701 | Plekhb1 | 9 | ACTGACAAAGCTG | 29 | CCTTGGCACCCGAGAATTCCAACTG |
| | | | TACTGAGTATGA | | ACAAAGCTGTACTGAGTATGA |
| ENSMUSG00000037625 | Cldn11 | 10 | GTCTCCATTCTGTT | 30 | CCTTGGCACCCGAGAATTCCAGTCT |
| | | | AGAGACCATGA | | CCATTCTGTTAGAGACCATGA |
| ENSMUSG00000019986 | Ahi1 | 11 | ATACAGGGTGGC | 31 | CCTTGGCACCCGAGAATTCCAATAC |
| | | | ACTGAAAACTG | | AGGGTGGCACTGAAAACTG |
| ENSMUSG00000022489 | Pde1b | 12 | ACTGCCTCCTCCTC | 32 | CCTTGGCACCCGAGAATTCCAACTG |
| | | | TCTTGTAAATA | | CCTCCTCCTCTCTTGTAAATA |
| ENSMUSG00000020262 | Adarb1 | 13 | ATGAATGTAACTC | 33 | CCTTGGCACCCGAGAATTCCAATGA |
| | | | AGCCAAGAAACG | | ATGTAACTCAGCCAAGAAACG |
| ENSMUSG00000036699 | Zcchc12 | 14 | TCTACCTTGTGAA | 34 | CCTTGGCACCCGAGAATTCCATCTA |
| | | | ACAATTGTCAGC | | CCTTGTGAAACAATTGTCAGC |
| ENSMUSG00000045573 | Penk | 15 | GTGGTCTAGATAA | 35 | CCTTGGCACCCGAGAATTCCAGTGG |
| | | | CTACACTGCCTG | | TCTAGATAACTACACTGCCTG |
| ENSMUSG00000026826 | Nr4a2 | 16 | ATTTCTAGTACGG | 36 | CCTTGGCACCCGAGAATTCCAATTT |
| | | | CACATGAGATGA | | CTAGTACGGCACATGAGATGA |
| ENSMUSG00000068696 | Gpr88 | 17 | TGGACCAAGAATG | 37 | CCTTGGCACCCGAGAATTCCATGGA |
| | | | GTAAGAACATTTG | | CCAAGAATGGTAAGAACATTTG |
| ENSMUSG00000021948 | Prkcd | 18 | ATAGAAAGCATGT | 38 | CCTTGGCACCCGAGAATTCCAATAG |
| | | | AGGAGACTGGTG | | AAAGCATGTAGGAGACTGGTG |
| ENSMUSG00000046321 | Hs3st2 | 19 | GACGACGATATCT | 39 | CCTTGGCACCCGAGAATTCCAGACG |
| | | | TTGAAAAGCACT | | ACGATATCTTTGAAAAGCACT |
| ENSMUSG00000064179 | Tnnt1 | 20 | GAAGTGAGACTG | 40 | CCTTGGCACCCGAGAATTCCAGAAG |
| | | | CCAGGACATGA | | TGAGACTGCCAGGACATGA |
| ENSMUSG00000033831 | Fgb | 21 | CCCTCAACTGTTC | 41 | CCTTGGCACCCGAGAATTCCACCCT |
| | | | ACTCTTAGAACT | | CAACTGTTCACTCTTAGAACT |

TABLE 3
Capture Analyte Sequences

| | | Captured | |
|---|---|---|---|
| Ensembl | Target | Sequence | |
| Identifier | Analyte | Identifier | Captured Sequence of Each Analyte |
| ENSMUSG00000041607 | Mbp | 42 | ACTGACACTGGAATAGGAATGTGATGGGCGTCGCACCCTCT |
| | | | GTAAATGTGGGAATGTTTGTAACTTGTGTTTGTATCTAATG |
| | | | TCGATCCCCTTAGGTGGTTGTACTATAATTCATTTTTGTTG |
| | | | TAAACTTCAGCCTAAGATAAATGTACATCTGCTTTTGTATG |
| | | | CACTCATTAAACATTGTAACAGACCAAAGATGGTGTACTAA |
| | | | TTGC |
| ENSMUSG00000057666 | Gapdh | 43 | CTCCAAGGAGTAAGAAACCCTGGACCACCCACCCCAGCAA |
| | | | GGACACTGAGCAAGAGAGGCCCTATCCCAACTCGGCCCCC |
| | | | AACACTGAGCATCTCCCTCACAATTTCCATCCCAGACCCC |
| | | | CATAATAACAGGAGGGGCCTAGGGAGCCCTCCCTACTCTC |
| | | | TTGAATACCATCAATAAAGTTCGCTGCACCC |
| ENSMUSG00000031425 | Plp1 | 44 | CTGCCCTCTGGGATGGATCTATAGATGGACTTTCCAAGCT |
| | | | CTCAGAAGCTGAGAGCATCTCCAGTTTATCAATTGAGCCC |
| | | | ATTGTTCTTAGCTCTCTCCCACATCATAAACCTTCTTTCT |
| | | | CTGAACAGAAAAGAGCTCTCTTTGTACTCAGATCAGCTAA |
| | | | AAATTAAAATACAGCAGTGTAAACAAGAAATTTTCTTACAA |
| ENSMUSG00000029580 | Actb | 45 | TTTTAATTTCTGAATGGCCGAGGTCTGAGGCCTCCCTTTTT |
| | | | TTTGTCCCCCCAACTTGATGTATGAAGGCTTTGGTCTCCCT |
| | | | GGGAGGGGGTTGAGGTGTTGAGGCAGCCAGGGCTGGCCTGT |
| | | | ACACTGACTTGAGACCAATAAAAGTGCACACCTTACCTTAC |
| | | | ACAAACA |
| ENSMUSG00000026223 | Itm2c | 46 | TCCTGCATGTTTTTACTGATGTTCGTGCTGGCTGCCCTCAG |
| | | | CCCTGAGTCTGGGAGAGGCTTTGGTGCCTCGGGTCAGAC |
| | | | TTGGGTGCTCCATGGTAGTGGAGCCCTTAAATGCTTTGTAT |
| | | | ATTTTCTCTATTAGATCTCTTTTCAGAAGTGTCTGTAGAAAA |
| | | | TTA AAAAAAAACA |
| ENSMUSG00000054459 | Vsnl1 | 47 | CTGGAATTTGCAGAATGACTGGAAGTGGGGAAAGTCACTG |
| | | | TCCAACTTATCATCCAGCCCCTCCTTCCCCAAAGATCAATA |
| | | | TGGCATGTAATATTTAAAGAAAACAGGAGATTTGTTCATTC |
| | | | TGGAAAACAATGCTCATTATGTGACAATAAACTTTATCTCA |
| | | | GTGTGACTTTGGTGCCAACAA |
| ENSMUSG00000060802 | B2m | 48 | ATCATATGCCAAACCCTCTGTACTTCTCATTACTTGGATGC |
| | | | AGTTACTCATCTTTGGTCTATCACAACATAAGTGACATACTT |
| | | | TCCTTTTGGTAAAGCAAAGAGGCCTAATTGAAGTCTGTCAC |
| | | | TGTGCCCAATGCTTAGCAATTCTCACCCCCAACCCTGTGG |
| | | | CTACTTCTGCTTTTGTTACT TTTACTAAAAATAAAAAACT |
| ENSMUSG00000030701 | Plekhb1 | 49 | ACTGACAAAGCTGTACTGAGTATGACCATATGATATTAAGT |
| | | | CGAGCAGAGGTCCCAGGAACCACAAGGCCAACCCTCCAA |
| | | | GCATCTTCTCGCAGTACCTTTGTTTTCACCAACCTCTCTTG |
| | | | TCATTTGTTGTGTCCTAATGCTACTTCTGAAGATAGCTGCA |
| | | | CCAATAAAATCTATGGCCTGTGGTT |
| ENSMUSG00000037625 | Cldn11 | 50 | GTCTCCATTCTGTTAGAGACCATGAAGCAGTATTGTTTAAC |
| | | | ATAAGTTGTACTGTTAAGTTTGGCTTCATGGGTGTAAACAC |
| | | | CAATGGTCTGTCAGTGTCTAAGACTCTGGATACTGCAAGC |
| | | | TCCGTCCGGTGCATTTGTTCAGGTAAAATCTGTGCAATAAA |
| | | | ATAACAAAC TGTCTCCAAA |
| ENSMUSG00000019986 | Ahi1 | 51 | ATACAGGGTGGCACTGAAAACTGCTGAAGCCCACAGCCCT |
| | | | CAGCCCCGAAGGCTCAGCGGCCAGTACCAGCGGCCTTGC |
| | | | TGTGGTTGTGTGTCTGCGTCCCTGGCACACTTGGGCAGGC |
| | | | AGCTCAGTCACTCTGAACTTGCTTCTCTTCTGTCTTGTGAG |
| | | | TGTGCTGCTTGAGACAATGAAAAATAAAGCTGTGTTCCTAC |
| | | | CCCTTCA |
| ENSMUSG00000022489 | Pde1b | 52 | ACTGCCTCCTCCTCTCTTGTAAATACATGCATTTGTACAGT |
| | | | GGGCCCTGTTCTTGTGAAGTCCATCTCCATGGTCATTAGA |
| | | | CCTGCCACTCTGAACCGCATGTGACTCCCCCATGCTCTTG |
| | | | GTCTCCCAGGCCCCTGCTATAGCCAGAGATCAATAAAGAA |
| | | | GGGAGACCGGC |
| ENSMUSG00000020262 | Adarb1 | 53 | ATGAATGTAACTCAGCCAAGAAACGTGTTGCTAAGATACAA |
| | | | TCCTCAGTGTTCTCTGTATGTATATTTCTGTATATACCACAT |
| | | | GTTACAGCCTGCATGAGCTTCCTCACACCAAGCCCAGCCG |
| | | | GCACTGAGCATGAGATGCTGTTACATGTAGACAAAGGACT |
| | | | GAGATGTTCTCAATAAAGACTAAGACGTTTCACTATG |
| ENSMUSG00000036699 | Zcchc12 | 54 | TCTACCTTGTGAAACAATTGTCAGCCCTTTGGTGCCTATCC |
| | | | TTCTAAATATTTCTCTATATCTGTGTTCCTAGATTAGAAATA |
| | | | TGTATAGACGAAAGTGATCAAATAGAAGTGTTGTTCTATAT |
| | | | GCTGTATTTTTTCACCAAAACGTATGTTGTGGCCTTCTTTG |
| | | | TCAATAAATATATACATATATGTCAGCATCT |
| ENSMUSG00000045573 | Penk | 55 | GTGGTCTAGATAACTACACTGCCTGAAAGCTGTGATTTTAG |
| | | | GGTCTGTGTTCTTTTGAGTCTTGAAGCTCAGTATTGGTCTC |
| | | | TTATGGCTATGTTGTTATCAATAGTTTGTTACCTCATCTCTC |
| | | | CTGACGAAACATCAATAAATGCTTATTTGTATATAAATATAA |
| | | | TAAACCCGTGACCCCAACTGCACAATG |
| ENSMUSG00000026826 | Nr4a2 | 56 | ATTTCTAGTACGGCACATGAGATGAGTCACTGCCTTTTTTT |
| | | | CTATGGTGTACGACAGTTAGAGATGCTGATTTTTTTTTCCT |
| | | | GATAAATTCTTTCTTTAAGAAAGACAATTTTAATGTTTACAA |
| | | | CAATAAACCACGTAAATGAACAGAA |
| ENSMUSG00000068696 | Gpr88 | 57 | TGGACCAAGAATGGTAAGAAGATTTGTATTTTTGAAAAAAT |
| | | | TGGGAGACACGGGAAACAGATATTTTATAGCAAGGCAAAA |
| | | | TAAAATAAATATGTTTGTCACTAACAATACGTTGGCAGTCAT |
| | | | GTCATTAACCAAACTGTGTGCATGTGTCATTTTTCTCTTAC |
| | | | GAAGATTTCTTCTGTTTCCAGTTTCCTGGATTCAGATATTTA |
| | | | ATTAAAGTTTCCATAATGCTTC |
| ENSMUSG00000021948 | Prkcd | 58 | ATAGAAAGCATGTAGGAGACTGGTGATGTGTTGACCTTTTT |
| | | | AAAAAAAAACATATGTATATGTGTGTATATATATATACACAC |
| | | | ATATACATATATATGTGTATGTATGTACGTATGTATATATAT |
| | | | ATGACCAAAAGAAAAGAAGAGCACAAGCTGTCTGAACCAC |
| | | | AGGTTCTTTT ATGTGTGTCTAAATAAACACTGAATGGTACC |
| ENSMUSG00000046321 | Hs3st2 | 59 | GACGACGATATCTTTGAAAAGCACTCTGTGACTCTCCCTG |
| | | | CTCCCTGCGGACAAAAGCACATAATCTGCTGTTACGGGTA |
| | | | CTTTCTTCACGCGAGCTTTCATGTTCAGCATGCACGGGAT |
| | | | CATGCTTGTCCATGTGAAATAAATATGGCTCTCTOGTGTCC |
| | | | TTAACA |
| ENSMUSG00000064179 | Tnnt1 | 60 | GAAGTGAGACTGCCAGGACATGACOTGOTGTGTGGAGCC |
| | | | CAGGAGCCACTGGAGCGTGTCCCATCTGTAACTCAAAATA |
| | | | AAGTGCCCCCAGGCATCTGCTTAAGTTCTTCAGGGTTGTT |
| | | | ATTATGTGGGTTGATCGACATCTCCATACTGCCTGGGAGA |
| | | | GCCATCAACTGTCATTAAAGAGAGTTCAAGTTC |
| ENSMUSG00000033831 | Fgb | 61 | CCCTCAACTGTTCACTCTTAGAACTTTCTAAAAGCCTAAGG |
| | | | AAATTGCTCACATTTTGACAATGAATACTAGCCAACCATCT |
| | | | GTTTTGCTTCCCTAAGAAGCATTTTTTTCAACTTTTATTCC |
| | | | AGTCTGAGAAGCTTGTAATTTCTAAACATTGAATGAATAAA |
| | | | CTCAAAGAATTGCCAATGCC |

### Example 2: Varying Conditions for Second Strand Synthesis

Given the ability to detect a cohort of genes from Example 1, optimal conditions for second strand synthesis were determined. After analyte capture, capture probe extension, and analyte digestion as described in Example 1, second strand cDNA synthesis was performed for 30 minutes on three-day old tissue sections. The amplification reaction was carried out for 22 cycles. Table 4 shows four experimental condition groups (Groups A-D) that were tested while varying the primer concentration and whether the tissue was removed.

TABLE 4
Experimental Conditions

| | | Primer |
|---|---|---|
| Group | Tissue Removal | Concentration |
| A | Triton 0.5%, no tissue removal | 1 μM primer pool |
| B | Triton 0.5%, tissue removal | 1 μM primer pool |
| C | Triton 1%, no tissue removal | 10 nM primer pool |
| D | Triton 1%, tissue removal | 10 nM primer pool |

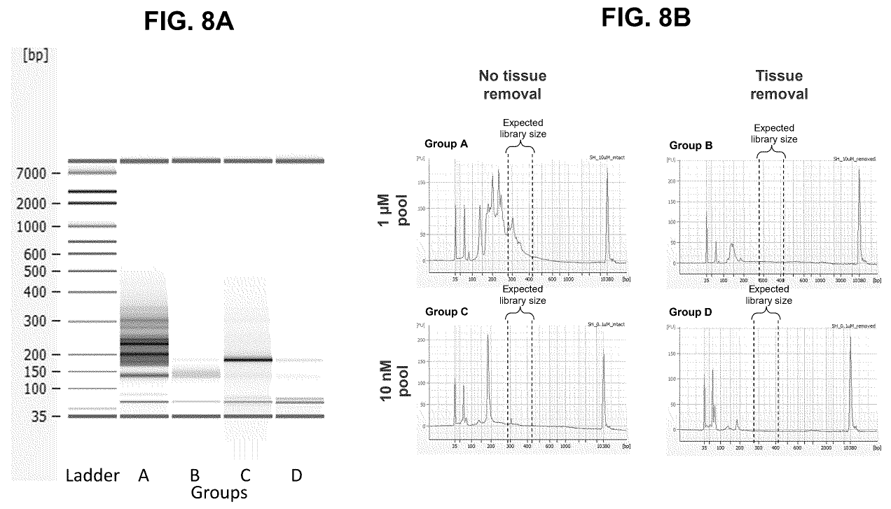

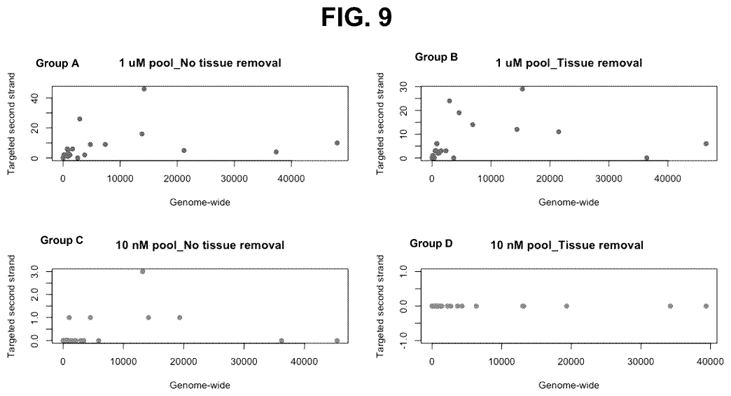

As shown in FIGS. 8A-8B and FIG. 9, analytes were most readily detected in Group A (Triton 0.5%, no tissue removal; 1 μM primer pool). Consistent with these data, Table 5 shows the results of the experiment, indicating that the highest number of detected genes occurred in Group A, in which (i) no tissue was removed from the array and (ii) the primer pool concentration was 1 μm.

TABLE 5
Groups A-D Results

| | Number of | | | | | | | |
|---|---|---|---|---|---|---|---|---|
| | Spots | Mean | Median | | | | | Total |
| | under | Reads | Genes | Number of | Valid | Valid | Seq | Genes |
| Group | Tissue | per Spot | per Spot | Seq Reads | Barcodes | UMIs | Saturation | Detected |
| A | 2944 | 5338 | 3 | 15,714,831 | 94.80% | 99.90% | 97.00% | 2119 |
| B | 2933 | 2923 | 2 | 8,574,525 | 67% | 99.90% | 98.20% | 1710 |
| C | 2892 | 3803 | 2 | 10,998,407 | 95.50% | 99.80% | 89.60% | 316 |
| D | 2848 | 1099 | 0 | 3,131,081 | 88.30% | 98.90% | 96.80% | 262 |

These data demonstrate a proof of concept that one can optimize the conditions (e.g., with or without tissue removal; primer concentration) to increase analyte detection. Because of the results in Group A, additional analysis was performed on this Group. In particular, a sequencing comparison was performed looking at detection of the original cDNA compared to detection of the targeted second strand (TSS). As shown in Tables 6 and 7, sequencing results for the targeted second strand indicated that Group A (compared to the original cDNA) had an increase in sequencing saturation, an increase in reads mapped confidently to intergenic regions, an increase in cDNA PCR Duplication, and a decrease in the fraction of UMI counts that were mapped to ribosomal protein. Taken together, these data provide proof of concept that the methods using second strand amplification using an adaptor and a primer as disclosed herein readily target and detect sequences of interest.

TABLE 6
Group A Results with and without Second Strand Synthesis using Adaptor and Primer

| | | | | | | Reads | | |
|---|---|---|---|---|---|---|---|---|
| | | | | | | Mapped | Reads | Reads |
| | Mean | | | | Reads | Confidently | Mapped | Mapped |
| | Reads | Reads Mapped | | | Mapped | to | Confidently | Confidently |
| | per | Confidently to | Number | Sequencing | to | Intergenic | to Intronic | to Exonic |
| Name | Cell | Transcriptome | of Reads | Saturation | Genome | Regions | Regions | Regions |
| V10J14_005_A | 69207 | 28.80% | 203606932 | 72.20% | 60.70% | 14.00% | 10.00% | 29.60% |
| (“cDNA”) | | | | | | | | |
| V10J14_005_A_TSS | 5338 | 3.50% | 15714831 | 97.00% | 54.20% | 34.40% | 6.10% | 4.00% |
| (“Targeted Second | | | | | | | | |
| Strand”) | | | | | | | | |

TABLE 7
Group A Results with and without Second Strand Synthesis using Adaptor and Primer

| | | cDNA PCR | | | | Fraction | |
|---|---|---|---|---|---|---|---|
| | | Duplication | | | Median | Ribosomal | |
| | Fraction | (5k raw | Median | Total | UMI | Protein | Fraction |
| | reads | reads per | Genes | Genes | Counts | UMI | Mitochondrial |
| Name | unmapped | cell) | per Cell | Detected | per Cell | Counts | UMI Counts |
| V10J14_005_A | 39.30% | 19.30% | 1922 | 18355 | 4680 | 1.60% | 23.20% |
| (“cDNA”) | | | | | | | |
| V10J14_005_A_TSS | 45.80% | 96.90% | 3 | 2119 | 3 | 0.40% | 25.30% |
| (“Targeted Second | | | | | | | |
| Strand”) | | | | | | | |

### Example 3: Using Hot-Start AMP Mix to Increase Priming Specificity of Second Strand Synthesis

Using a fresh mouse brain tissue sample, parameters were adjusted to test whether using Hot Start Taq DNA Polymerase would affect target-specific detection using a primer comprising an adaptor as described herein. Briefly, a fresh mouse brain tissue sample was sectioned and placed on an array comprising a plurality of capture probes. After permeabilization, analyte capture, capture probe extension, and second strand synthesis was performed for 15 minutes followed by 22 cycles of PCR.

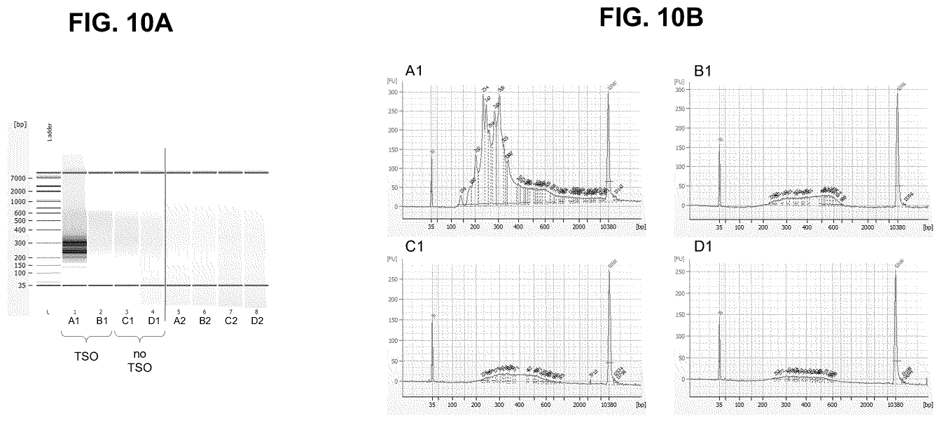

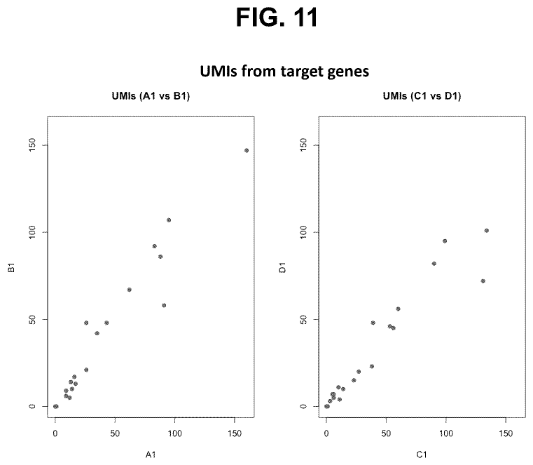

Second strand synthesis was performed using either second strand mix (i.e., the same buffer from Examples 1 and 2), or using Hot Start AMP Mix Buffer. See Table 7. As shown in FIGS. 10A-10B, analyte detection was observed in the group treated with second strand mix (i.e., the same condition from Examples 1 and 2), but was not readily detected in any group using Hot Start AMP Mix Buffer. Further, detection of UMIs from each group treated with second strand (with or without TSO) showed consistent detection. See FIG. 11.

TABLE 8
Hot Start

| | | With or | | | With or |
|---|---|---|---|---|---|
| | | without | | | without |
| Sample | Condition | TSO | Sample | Condition | TSO |
| A1 | Second | TSO | A2 | Hot Start | TSO |
| | Strand Mix | | | AMP Mix | |
| B1 | Second | TSO | B2 | Hot Start | TSO |
| | Strand Mix | | | AMP Mix | |
| C1 | Second | No TSO | C2 | Hot Start | No TSO |
| | Strand Mix | | | AMP Mix | |
| D1 | Second | No TSO | D2 | Hot Start | No TSO |
| | Strand Mix | | | AMP Mix | |

The experimental settings were modified to adjust the temperature either before extension (to 98° C.) or during extension (to 65° C.). See Table 9.

TABLE 9
Hot-Start Conditions

| Sample | Condition | Buffer | Sample | Condition | Buffer |
|---|---|---|---|---|---|
| A1 | Pre-heat | Hot- | A2 | Thermo | Hot- |
| | 98° C.; | start | | mixer | start |
| | 65° C. | Amp mix | | 98° C.; | Amp |
| | extension | | | 3′-65° C. | mix |
| B1 | Pre-heat | | B2 | Thermo | |
| | 98° C.; | | | mixer | |
| | 65° C. | | | 98° C.; | |
| | extension | | | 3′-65° C. | |
| C1 | 65° C. | Second- | | | |
| | extension | strand | | | |
| D1 | 65° C. | reagent | | | |
| | extension | | | | |

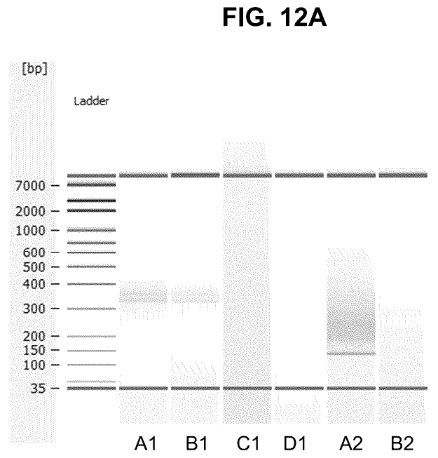

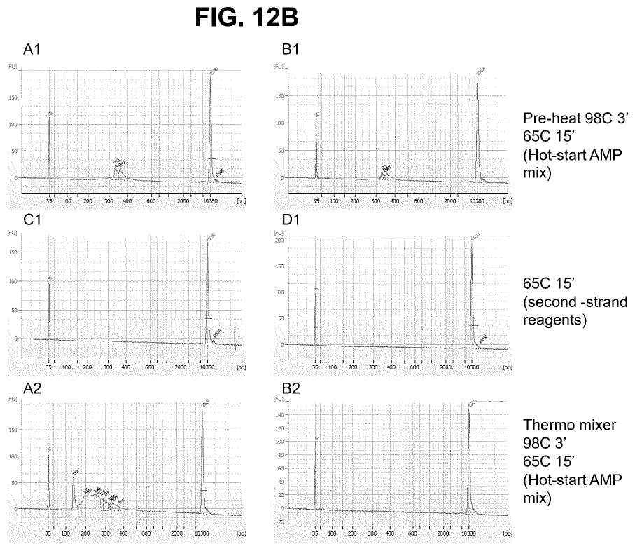

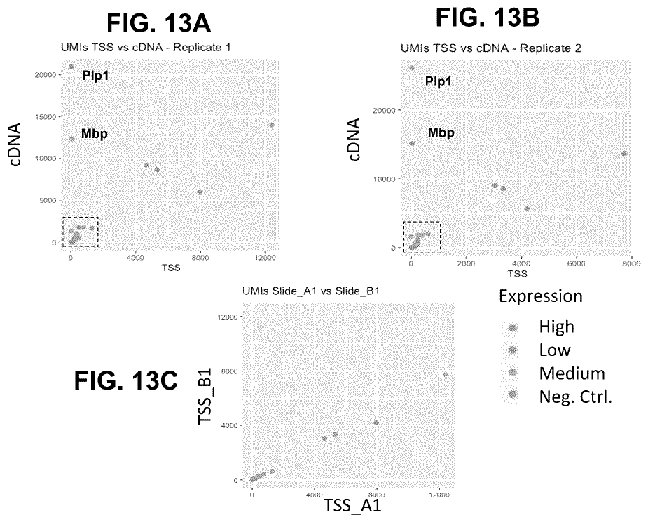

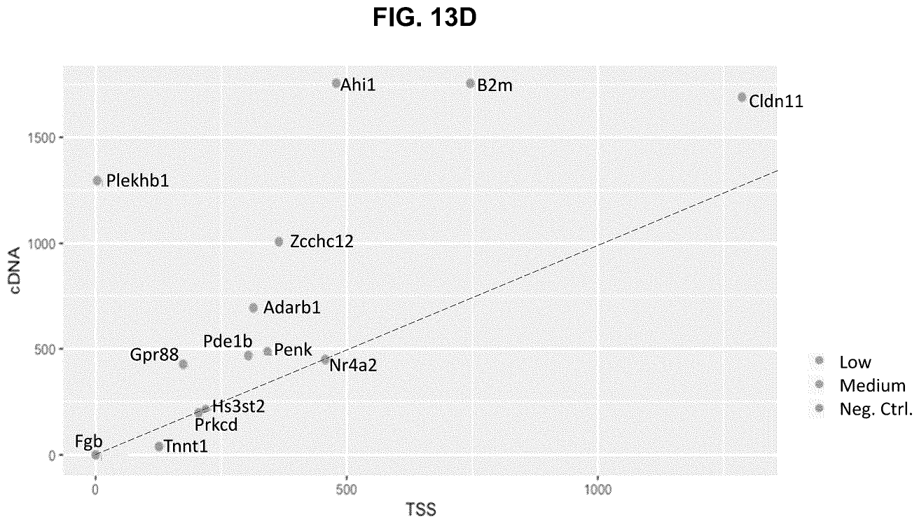

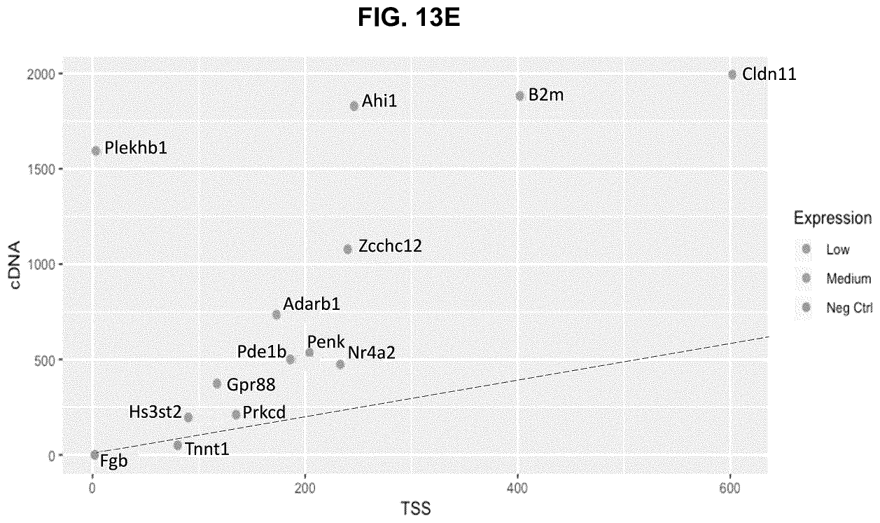

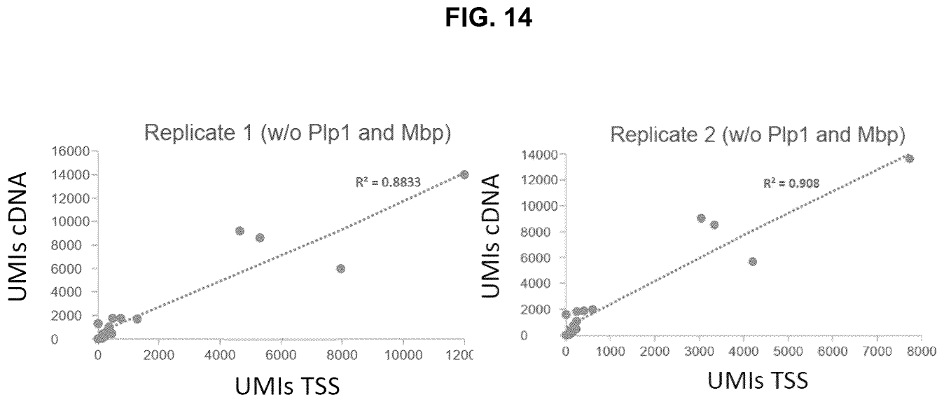

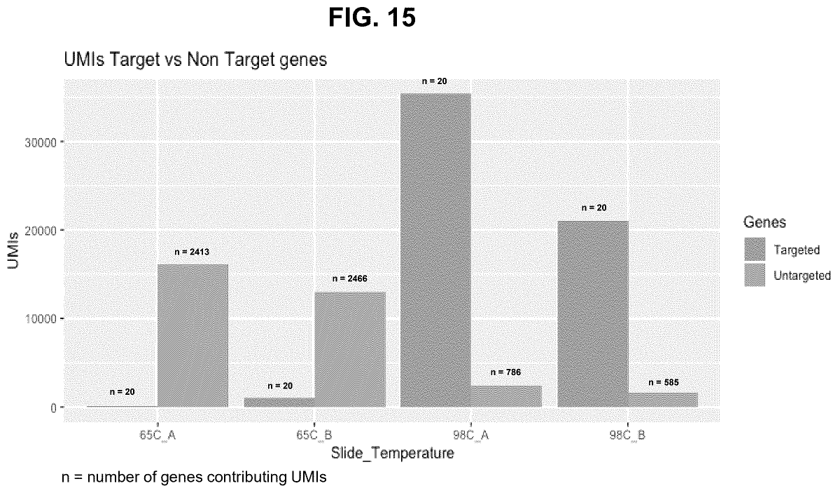

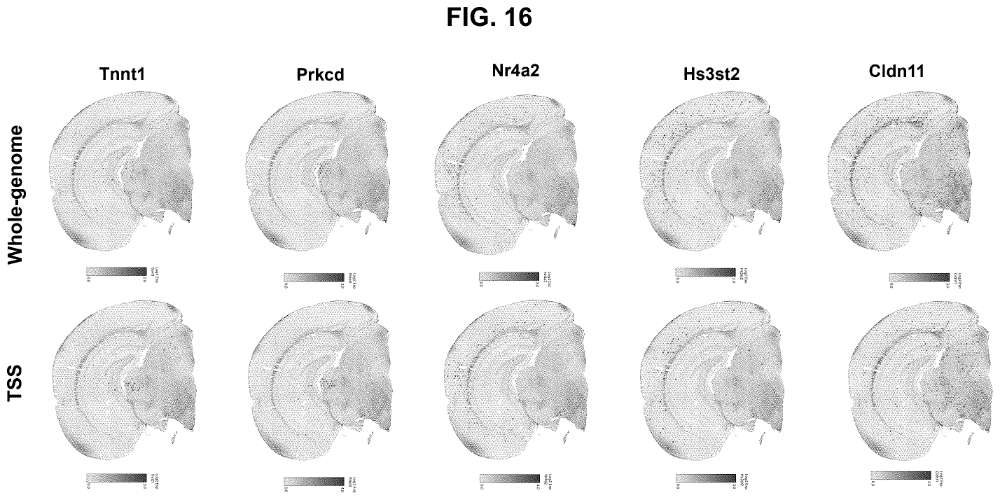

As shown in FIGS. 12A-12B, analyte detection was observed in the groups pre-heated to 98° C. (A1, B1, and A2) while samples without Hot-Start Amp Mix displayed very little analyte detection. Further, FIGS. 13A-13E and 14 show only minor variance when comparing analytes detected using second strand synthesis (TSS) compared to whole genome detection (cDNA). In addition, as shown in FIG. 15 and in Tables 10 and 11 below, using Hot-Start Amp Mix resulted in an increases in UMIs specific to targeted genes, confidently mapped reads, and sequencing saturation as well as a decrease in detection of off-target ribosomal protein analytes. Finally, the spatial patterns of particular targeted genes (Tnnt1, Prkcd, Nr4a2, Hs3st2, and Cldn11) were determined, showing specific expression using the second-synthesis methods described herein compared to methods detection of analytes without second-strand synthesis. See FIG. 16.

Taken together, these data show that using a Hot-Start Amp Mix Buffer while increasing the temperature before and during second strand synthesis can increase specific detection of target analytes while decreasing off-target capture.

TABLE 10
Hot-Start Amp Mix Results

| | | | | | | Reads | Reads | Reads | |
|---|---|---|---|---|---|---|---|---|---|
| | | Reads | | | Reads | Mapped | Mapped | Mapped | |
| | Mean | Mapped | | | Mapped | Confidently | Confidently | Confidently | |
| | Reads | Confidently | | | Confidently | to | to | to | Fraction |
| | per | to | Number of | Sequencing | to | Intergenic | Intronic | Exonic | reads |
| Sample | Cell | Transcriptome | Reads | Saturation | Genome | Regions | Regions | Regions | unmapped |
| 95_A_cDNA | 30149 | 32.90% | 93.521.696 | 63.60% | 60.90% | 16.70% | 10.50% | 33.80% | 33.50% |
| 95_B_cDNA | 38731 | 31.90% | 121.537.022 | 70.90% | 60.30% | 16.20% | 11.20% | 32.80% | 34.20% |
| 95_A_TSS | 16348 | 81.80% | 49.879.158 | 99.90% | 86.10% | 1.40% | 2.10% | 82.60% | 11.10% |
| 95_B_TSS | 9593 | 79.10% | 29.269.288 | 99.90% | 84.40% | 1.60% | 2.90% | 80.00% | 12.50% |

TABLE 11
Hot-Start Amp Mix Results

| | | | | | Median | Median | | |
|---|---|---|---|---|---|---|---|---|
| | cDNA PCR | | | | genes per | UMI | | |
| | Duplication | | | Median | cell (5k | counts | Fraction | |
| | (5k raw | Median | Total | UMI | raw | per cell | ribosomal | Fraction |
| | reads per | Genes | Genes | Counts | reads per | (5k raw | protein | mitochondrial |
| Sample | cell) | per Cell | Detected | per Cell | cell) | reads per cell) | UMI counts | UMI counts |
| 95_A_cDNA | 24.00% | 1308 | 17676 | 2990 | 531 | 1034 | 1.60% | 22.70% |
| 95_B_cDNA | 26.20% | 1366 | 17789 | 3158 | 530 | 1032 | 1.60% | 24.00% |
| 95_A_TSS | 99.80% | 4 | 761 | 10 | 4 | 7 | 0.10% | 0.20% |
| 95_B_TSS | 99.90% | 2 | 571 | 5 | 2 | 4 | 0.30% | 0.20% |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising:
(a) capturing a nucleic acid from the biological sample on an array comprising a plurality of capture probes, wherein a capture probe of the plurality of capture probes comprises a capture domain and a spatial barcode;
(b) extending an end of the capture probe using the nucleic acid as a template, thereby generating an extended capture probe hybridized to the nucleic acid;
(c) delivering a plurality of primers to the array, wherein each primer of the plurality of primers comprises in a 5′ to a 3′ direction: (i) a sequencing adaptor and (ii) a gene-specific sequence that binds to the extended capture probe; and
(d) extending a 3′ end of the primer from the plurality of primers bound to the extended capture probe using the extended capture probe as a template, thereby generating a DNA molecule hybridized to the extended capture probe.

2. The method of claim 1, further comprising determining (i) all or a part of the sequence of the DNA molecule, or a complement thereof, and (ii) the sequence of the spatial barcode, or a complement thereof, and using the determined sequences of (i) and (ii) to identify a location of the nucleic acid in the biological sample.

3. The method of claim 1, wherein the extending in step (b) comprises contacting the capture probe hybridized to the nucleic acid with a reverse transcriptase.

4. The method of claim 1, further comprising, between steps (b) and (c), digesting the nucleic acid hybridized to the extended capture probe.

5. The method of claim 4, wherein the digesting comprises contacting the nucleic acid with a RNAase H or a functional equivalent thereof.

6. The method of claim 1, wherein the extending in step (d) comprises contacting the primer bound to the extended capture probe with a DNA polymerase.

7. The method of claim 1, further comprising releasing the DNA molecule from the extended capture probe, wherein the releasing the DNA molecule comprises heating the DNA molecule to de-hybridize the DNA molecule from the extended capture probe.

8. The method of claim 2, wherein the determining in step (e) comprises sequencing (i) all or a part of the sequence of the DNA molecule or a complement thereof, and (ii) the spatial barcode or a complement thereof.

9. The method of claim 1, wherein the nucleic acid is an mRNA molecule.

10. The method of claim 1, wherein the capture domain comprises a poly(T) sequence.

11. The method of claim 1, wherein the capture probe further comprises one or more of a functional domain, a unique molecular identifier, and a cleavage domain.

12. The method of claim 1, wherein the capturing in step (a) comprises permeabilizing the biological sample using a permeabilization agent, wherein the permeabilization agent comprises proteinase K or pepsin, thereby releasing the nucleic acid from the biological sample.

13. The method of claim 1, wherein the biological sample is a tissue section.

14. The method of claim 13, wherein the tissue section is a formalin-fixed paraffin-embedded tissue section or a fresh frozen tissue section.

15. The method of claim 1, wherein the method further comprises imaging the biological sample.

16. The method of claim 1, wherein the plurality of primers is a primer pool, wherein the primer pool is at a concentration of 1 μM.

17. The method of claim 1, wherein abundance of the nucleic acid is increased by at least 10% compared to the method of claim 1 that does not utilize the plurality of primers of step (c).

18. The method of claim 1, wherein the plurality of primers comprises primers that amplify the transcriptome of the biological sample.

19. A composition comprising:
(a) an array comprising a plurality of capture probes, wherein a capture probe of the plurality of capture probes comprises a capture domain and a spatial barcode;
(b) a plurality of primers, wherein each primer of the plurality of primers comprises in a 5′ to a 3′ direction: (i) a sequencing adaptor and (ii) a gene-specific sequence; and
(c) a polymerase or reverse transcriptase.

20. The composition of claim 19, wherein the sequencing adaptor comprises SEQ ID NO:1.
